SEQUENCE LISTING (73 sequences)

SEQ ID NO: 1 - protein, 7 aa, Artificial
  Synthetic
ARRPKLD

SEQ ID NO: 2 - protein, 10 aa, Artificial
  Synthetic
ARPPKGVNWT

SEQ ID NO: 3 - protein, 10 aa, Artificial
  Synthetic
ARLPQVELSA

SEQ ID NO: 4 - protein, 10 aa, Artificial
  Synthetic
ARAPGVMPTT

SEQ ID NO: 5 - protein, 7 aa, Artificial
  Synthetic
ARMPPRS

SEQ ID NO: 6 - protein, 7 aa, Artificial
  Synthetic
ARRPATL

SEQ ID NO: 7 - protein, 9 aa, Artificial
  Synthetic
ARRPAVAFE

SEQ ID NO: 8 - protein, 7 aa, Artificial
  Synthetic
ARRPPQM

SEQ ID NO: 9 - protein, 7 aa, Artificial
  Synthetic
ARQPAHF

SEQ ID NO: 10 - protein, 7 aa, Artificial
  Synthetic
ARKPVFQ

SEQ ID NO: 11 - protein, 10 aa, Artificial
  Synthetic
ARNPTLGNSS

SEQ ID NO: 12 - protein, 7 aa, Artificial
  Synthetic
ARPPRST

SEQ ID NO: 13 - protein, 7 aa, Artificial
  Synthetic
ARSPRVK

SEQ ID NO: 14 - protein, 10 aa, Artificial
  Synthetic
ARSPHVTPIA

SEQ ID NO: 15 - protein, 7 aa, Artificial
  Synthetic
ARSPISP

SEQ ID NO: 16 - protein, 7 aa, Artificial
  Synthetic
ARYPVTM

SEQ ID NO: 17 - protein, 10 aa, Artificial
  Synthetic
ARTPSRTPVV

SEQ ID NO: 18 - protein, 7 aa, Artificial
  Synthetic
ARAPKMG

SEQ ID NO: 19 - protein, 7 aa, Artificial
  Synthetic
ARAPGVR

SEQ ID NO: 20 - protein, 10 aa, Artificial
  Synthetic
ARAPGPPRLA

SEQ ID NO: 21 - protein, 7 aa, Artificial
  Synthetic
ARAPKMG

SEQ ID NO: 22 - protein, 7 aa, Artificial
  Synthetic
ARAPYAS

SEQ ID NO: 23 - protein, 7 aa, Artificial
  Synthetic
ARMPQYT

SEQ ID NO: 24 - protein, 10 aa, Artificial
  Synthetic
ARLPRAVVPL

SEQ ID NO: 25 - protein, 10 aa, Artificial
  Synthetic
SRNPGLLTVR

SEQ ID NO: 26 - protein, 10 aa, Artificial
  Synthetic
SRAPNSVQHD

SEQ ID NO: 27 - protein, 7 aa, Artificial
  Synthetic
SRAPVAP

SEQ ID NO: 28 - protein, 10 aa, Artificial
  Synthetic
SRLPSAGTFQ

SEQ ID NO: 29 - protein, 7 aa, Artificial
  Synthetic
SRRPAIM

SEQ ID NO: 30 - protein, 7 aa, Artificial
  Synthetic
SRRPVWF

SEQ ID NO: 31 - protein, 7 aa, Artificial
  Synthetic
SRRPQLP

SEQ ID NO: 32 - protein, 10 aa, Artificial
  Synthetic
SRRPAFVVRV

SEQ ID NO: 33 - protein, 10 aa, Artificial
  Synthetic
SRRPGLSHAA

SEQ ID NO: 34 - protein, 7 aa, Artificial
  Synthetic
SRRPLVV

SEQ ID NO: 35 - protein, 7 aa, Artificial
  Synthetic
SRQPTSL

SEQ ID NO: 36 - protein, 10 aa, Artificial
  Synthetic
SRSPRVVEGL

SEQ ID NO: 37 - protein, 7 aa, Artificial
  Synthetic
SRSPLVV

SEQ ID NO: 38 - protein, 7 aa, Artificial
  Synthetic
SRYPVVS

SEQ ID NO: 39 - protein, 7 aa, Artificial
  Synthetic
SRTPPLL

SEQ ID NO: 40 - protein, 10 aa, Artificial
  Synthetic
SRSPRLALPT

SEQ ID NO: 41 - protein, 7 aa, Artificial
  Synthetic
SRSPVMS

SEQ ID NO: 42 - protein, 7 aa, Artificial
  Synthetic
SRYPLEL

SEQ ID NO: 43 - protein, 7 aa, Artificial
  Synthetic
SRWPGSV

SEQ ID NO: 44 - protein, 7 aa, Artificial
  Synthetic
SRPPART

SEQ ID NO: 45 - protein, 10 aa, Artificial
  Synthetic
SRAPLLRPII

SEQ ID NO: 46 - protein, 7 aa, Artificial
  Synthetic
SRAPVAP

SEQ ID NO: 47 - protein, 10 aa, Artificial
  Synthetic
SRAPLGSIAD

SEQ ID NO: 48 - protein, 10 aa, Artificial
  Synthetic
SRAPAVAGWK

SEQ ID NO: 49 - protein, 11 aa, Artificial
  Synthetic
SRAPAQKVFFG

SEQ ID NO: 50 - protein, 10 aa, Artificial
  Synthetic
SRAPSNVERM

SEQ ID NO: 51 - protein, 10 aa, Artificial
  Synthetic
SRAPSTLAHV

SEQ ID NO: 52 - protein, 10 aa, Artificial
  Synthetic
SRAPSPSYRQ

SEQ ID NO: 53 - protein, 7 aa, Artificial
  Synthetic
SRMPGSV

SEQ ID NO: 54 - protein, 7 aa, Artificial
  Synthetic
SRMPLPV

SEQ ID NO: 55 - protein, 10 aa, Artificial
  Synthetic
SRMPTLMSGL

SEQ ID NO: 56 - protein, 10 aa, Artificial
  Synthetic
SRLPEVVLGQ

SEQ ID NO: 57 - protein, 7 aa, Artificial
  Synthetic
SRLPART

SEQ ID NO: 58 - protein, 10 aa, Artificial
  Synthetic
SRLPVSATLA

SEQ ID NO: 59 - protein, 10 aa, Artificial
  Synthetic
SRVPGRATAT

SEQ ID NO: 60 - protein, 7 aa, Artificial
  Synthetic
SRVPLGP

SEQ ID NO: 61 - protein, 10 aa, Artificial
  Synthetic
SRVPLGRASS

SEQ ID NO: 62 - protein, 7 aa, Artificial
  Synthetic
SRVPSDV

SEQ ID NO: 63 - protein, 7 aa, Artificial
  Synthetic
SRVPYQN

SEQ ID NO: 64 - protein, 10 aa, Artificial
  Synthetic
SRVPVRGVFQ

SEQ ID NO: 65 - protein, 7 aa, Artificial
  Synthetic
SRRPKLD

SEQ ID NO: 66 - protein, 4 aa, Artificial
  Synthetic
ARRP

SEQ ID NO: 67 - protein, 4 aa, Artificial
  Synthetic
SRAP

SEQ ID NO: 68 - protein, 4 aa, Artificial
  Synthetic
ARAP

SEQ ID NO: 69 - protein, 4 aa, Artificial
  Synthetic
SRVP

SEQ ID NO: 70 - protein, 4 aa, Artificial
  Synthetic
SRLP

SEQ ID NO: 71 - protein, 4 aa, Artificial
  Synthetic
ARLP

SEQ ID NO: 72 - protein, 4 aa, Artificial
  Synthetic
ARPP

SEQ ID NO: 73 - protein, 4 aa, Artificial
  Synthetic
SRRP

(19) United States
(12) Patent Application Publication
(10) Pub. No.: US 2009/0180958 A1
(43) Pub. Date: Jul. 16, 2009

(54) Diagnostic and therapeutic agents

(75) Inventors: Aki Koivistoinen, Helsinki (FI); Mathias Bergman, Ostersundom (FI); Hannu Elo, Helsinki (FI)

Correspondence Address: BUCHANAN, INGERSOLL & ROONEY PC, POST OFFICE BOX 1404, ALEXANDRIA, VA, 22313-1404, US

(73) Assignee: Karyon-CTT Ltd., Helsinki (FI)

(21) Appl. No.: 11/919,426

(22) PCT Filed: Apr. 25, 2006
(86) PCT No.: PCT/FI2006/050162

§ 371 Date: Oct. 26, 2007

Related U.S. Application Data
(60) Provisional application No. 60/674,697, filed on Apr. 26, 2005.

(30) Foreign Application Priority Data
Apr. 26, 2005 (FI) 20050437

(51) Int. Cl. A61K 49/00; C07K 7/64; C07K 5/00; A61K 38/07; A61K 38/12; A61K 38/08

(52) U.S. Cl. 424/9.1; 530/330; 530/329; 514/16; 514/18; 514/15; 530/328; 530/327

(57) ABSTRACT

The present invention relates to tumor targeting units comprising a peptide sequence X—R—Y—P—Zₙ, or a pharmaceutically or physiologically acceptable salt thereof. The invention further relates to tumor targeting agents comprising at least one targeting unit according to the present invention, directly or indirectly coupled to at least one effector unit. The present invention further relates to diagnostic or pharmaceutical compositions comprising at least one targeting unit or at least one targeting agent according to the present invention, and to the use of targeting units or targeting agents according to the present invention for the preparation of a medicament for the treatment of cancer or cancer related diseases, especially for the treatment of non-small cell lung cancer or its metastases.

### FIELD OF THE INVENTION

The present invention relates to targeting agents, especially to tumor targeting agents, such as lung tumor and especially to non-small cell lung cancer (NSCLC) targeting agents comprising at least one targeting unit and at least one effector unit, as well as to tumor targeting units and motifs, such as lung tumor and NSCLC targeting units and motifs. Further, the present invention concerns pharmaceutical and diagnostic compositions comprising such targeting agents or targeting units, and the use of such targeting agents and targeting units as pharmaceuticals or as diagnostic tools. The invention further relates to the use of such targeting agents and targeting units for the preparation of pharmaceutical or diagnostic compositions. Furthermore, the invention relates to kits for diagnosing or treating cancer, such as lung cancer and especially non-small cell lung cancer.

### BACKGROUND OF THE INVENTION

Malignant tumors are among the greatest health problems of man as well as animals, being one of the most common causes of death, also among young individuals. Available methods of treatment of cancer are quite limited, despite intensive research efforts during several decades. Although curative treatment, usually surgery in combination with chemotherapy and/or radiotherapy, is sometimes possible, malignant tumors still require a huge number of lives every year. In fact, curative treatment is rarely accomplished if the disease is not diagnosed early. In addition, certain tumor types can rarely, if ever, be cured.

There are various reasons for this very undesirable situation, the most important one clearly being the fact that most treatment schedules, except surgery, lack sufficient selectivity. Chemotherapeutic agents commonly used do not act on the malignant cells of the tumors alone but are highly toxic to other cells as well, especially to rapidly dividing cell types, such as hematopoietic and epithelial cells, resulting in highly undesirable side effects. The same applies to radiotherapy.

In addition, two major problems plague the non-surgical treatment of malignant solid tumors. Physiological barriers within tumors impede the delivery of therapeutics at effective concentrations to all cancer cells, and acquired drug resistance resulting from genetic and epigenetic mechanisms reduces the effectiveness of available drugs.

Also in the diagnosis of cancer and of metastases, including the follow-up of patients and the study of the effects of treatment on tumors and metastases, reliable, sensitive and more selective methods and agents would be a great advantage. All methods currently in use, such as nuclear magnetic resonance imaging, X-ray methods, histological staining methods still lack agents that are capable of targeting an entity for detection specifically or selectively to tumor tissues, metastases or tumor cells and/or to tumor endothelium.

Lung cancer is the leading cause of cancer related mortality in both men and woman. Non-small cell lung cancer accounts around 80% and small cell lung cancer 20% of all lung cancers. It has been estimated that only 10% of the diagnosed lung cancer patients live more than five years. Often, at the moment of diagnosis the cancer has already spread so that surgical treatment, the only effective treatment, is not possible. In addition, patients whose cancer is surgically at a curable stage often have some other disease that makes surgical operation impossible. Early diagnosis is essential for successful treatment of non-small cell lung cancer (NSCLC). So far, early diagnosis is problematic and only spiral computer tomography has given satisfying results. However, as a method spiral CT is expensive and as a screening test impractical.

The long-term survival of patients undergoing conventional therapies (surgery, chemotherapy and radiation therapy) is poor. Current therapeutic agents such as the mitosis inhibitors (taxanes, such as paclitaxel and docetaxel), anti-metabolites (gemsitabine), vinca alkaloids (vinorelbine), and topoisomerase inhibitors (irinotecane) used in treatment of advanced NSCLC in combination with platinum containing drugs have reached a threshold of therapeutic effectiveness.

Monoclonal antibodies specific to cells of lung tumors have show clinical promise as targeted agents for the treatment of lung cancer. However, there are some major limitations in antibody-targeted therapy based on two facts: large size and non-specific uptake of the antibody molecules by the liver and the reticuloendothelial system. The large size results in poor tumor penetration of antibody pharmaceuticals and causes often immune response, whereas non-specific uptake by the liver and the reticuloendothelial system results in dose-limiting toxicity to the liver and bone marrow.

Targeting peptides are excellent alternative for targeted treatment of human cancers, and due to relatively small size they may overcome some of the problems with antibody targeting. Advantages of peptides are: greater stability—peptides can be stored at room temperature for weeks; lower manufacturing costs (synthetic production versus recombinant production); rapid pharmacokinetics; excretion route that can be modified; and higher activity per mass of final targeting agent.

There are numerous publications disclosing peptides homing to different cell and tissue types. Some of these are claimed to be useful as cancer targeting peptides. Among the earliest identified homing peptides described are the integrin and NGR-receptor targeting peptides described by Ruoslahti et al., in e.g., U.S. Pat. No. 6,180,084.

International Patent publication WO 00/12738 discloses targeted adenovirus vectors for delivery of heterologous genes. The disclosed vectors are described as containing peptide sequences, such as NQNSRRPSRA, targeting a urokinase-type plasminogen activator receptor (UPAR).

International Patent publication WO 02/020822 discloses a biopanning method for identifying selectively binding peptides, exemplified by e.g., CSRRPEVVC, which is a cyclic peptide-claimed to be targeting mesenchymal stem cells.

No publications disclosing peptides specifically targeting NSCLC cells have been identified. Thus, there is a need for targeting agents useful in diagnosis and therapy of NSCLC.

### BRIEF DESCRIPTION OF THE INVENTION

The present invention relates to tumor targeting units, targeting to lung cancer and more specifically to non-small cell lung tumor, comprising a peptide sequence X—R—Y—P—Zₙ or a pharmaceutically or physiologically acceptable salt or derivative thereof, wherein X is alanine, serine or homoserine, or a structural or functional analogue thereof; R is arginine or homoarginine, or a structural or functional analogue thereof; Y is arginine, alanine, leucine, serine, valine or proline; P is proline, or a structural or functional analogue thereof; Z is any amino acid residue and each Zₙ may be different or similar or identical, and n is an integer from 0 to 7. The targeting units of the present invention may be linear or cyclic or form part of a cyclic structure. The invention further relates to tumor targeting agents comprising at least one targeting unit according to the present invention, directly or indirectly coupled to at least one effector unit. Preferably the effector unit is a directly or indirectly detectable agent or a therapeutic agent.

The present invention further relates to diagnostic or pharmaceutical compositions comprising at least one targeting unit or at least one targeting agent according to the present invention, and to the use of targeting units or targeting agents according to the present invention for the preparation of a medicament for the treatment of cancer or cancer related diseases, especially for the treatment of non-small cell lung cancer or its metastases.

The present invention further relates to methods for treating cancer or cancer related diseases by providing to a patient in need thereof a therapeutically effective amount of a pharmaceutical composition according to the present invention for treating non-small cell lung cancer or its metastases.

### BRIEF DESCRIPTION OF THE DRAWINGS

In the following the invention will be described in greater detail by means of preferred embodiments with reference to the attached drawings, in which

FIG. 1 shows the selective binding of NSCLC cell lines to a targeting agent;

FIG. 2 shows that the peptide of the present invention is non-toxic in vitro; and

FIG. 3 shows that the peptide of the invention is non-immunogenic.

### DETAILED DESCRIPTION OF THE INVENTION

It is an object of the present invention to provide novel tumor targeting agents that comprise at least one targeting unit and, optionally, at least one effector unit. In an important embodiment, the invention provides targeting units comprising at least one motif capable of targeting solid tumors of the lungs. As a specific embodiment, the present invention provides tumor targeting motifs and units that specifically target non-small cell lung cancer cells.

The targeting units according to the present invention, optionally coupled to at least one effector unit, are therapeutically and diagnostically useful, especially in the treatment and diagnosis of cancer, including metastases, preferably tumors and metastases of the lung. Furthermore the targeting agents according to the present invention are useful for cell removal, selection, sorting and enrichment.

It is a second object of this invention to provide pharmaceutical and diagnostic compositions comprising at least one targeting agent or at least one targeting unit comprising at least one motif according to the present invention. Such compositions may be used to destroy tumors or hinder their growth, or for the diagnosis of cancer.

As early diagnosis of metastases is very important for successful treatment of cancer, an important use of the targeting units and targeting agents of this invention is in early diagnosis of tumor metastases.

A third object of the present invention is to provide novel diagnostic and therapeutic methods and kits for the treatment and/or diagnosis of cancer, preferably cancer of the lung, including metastases.

The targeting units of this invention may be used as such or coupled to at least one effector unit.

For the purpose of this invention, the term “cancer” is used herein in its broadest sense, and includes any disease or condition involving transformed or malignant cells. In the art, cancers are classified into five major categories, according to their tissue origin (histological type): carcinomas, sarcomas, myelomas, and lymphomas, which are solid tumor type cancers, and leukemias, which are “liquid cancers”. The term cancer, as used in the present invention, is intended to primarily include all types of diseases characterized by solid tumors, including disease states where there is no detectable solid tumor or where malignant or transformed cells, “cancer cells”, appear as diffuse infiltrates or sporadically among other cells in healthy tissue.

The terms “amino acid” and “amino alcohol” are to be interpreted herein to include also diamino, triamino, oligoamino and polyamino acids and alcohols; dicarboxyl, tricarboxyl, oligocarboxyl and polycarboxyl amino acids; dihydroxyl, trihydroxyl, oligohydroxyl and polyhydroxyl amino alcohols; and analogous compounds comprising more than one carboxyl group or hydroxyl group and one or more amino groups.

By the term “peptide” is meant, according to established terminology, a chain of amino acids (peptide units) linked together by peptide bonds to form an amino acid chain. Peptides may be linear or cyclic as described below. For the purposes of the present invention, also compounds comprising one or more D-amino acids, beta-amino acids and/or other unnatural amino acids (e.g. amino acids with unnatural side chains) are included in the term “peptide”. For the purposes of the present invention, the term “peptide” is intended to include peptidyl analogues comprising modified amino acids. Such modifications may for example comprise the introduction or presence of a substituent;

the introduction or presence of an “extra” functional group such as an amino, hydrazino, carboxyl, formyl (aldehyde) or keto group, or another moiety; and the absence or removal of a functional group or other moiety. The term also includes analogues modified in the amino and/or carboxy termini, such as peptide amides and N-substituted amides, peptide hydrazides, N-substituted hydrazides, peptide esters, and their like, and peptides that do not comprise the amino-terminal —NH₂ group or that comprise e.g. a modified amino-terminal amino group or an imino or a hydrazino group instead of the amino-terminal amino group, and peptides that do not comprise the carboxy-terminal carboxyl group or comprise a modified group instead of it, and so on.

Some examples of possible reaction types that can be used to modify peptides, forming “peptidyl analogues”, are e.g., condensation and nucleophilic addition reactions as well as esterification, amide formation, formation of substituted amides, N-alkylation, formation of hydrazides, salt formation. Salt formation may be the formation of any type of salt, such as alkali or other metal salt, ammonium salt, salts with organic bases, acid addition salts etc. Peptidyl analogues may be synthesized either from the corresponding peptides or directly (via other routes).

The expression “structural or functional analogues” of the peptides of the invention is used to encompass compounds that do not consist of amino acids or not of amino acids alone, or some or all of whose building blocks are modified amino acids. Different types of building blocks can be used for this purpose, as is well appreciated by those skilled in the art. The function of these compounds in biological systems is essentially similar to the function of the peptides. The resemblance between these compounds and the original peptides is thus based on structural and functional similarities. Such compounds are called peptidomimetic analogues, as they mimic the function, conformation and/or structure of the original peptides and, for the purposes of the present invention, they are included in the term “peptide”.

A functional analog of a peptide according to the present invention is characterized by a binding ability with respect to the binding to tumors, tumor tissue, tumor cells or tumor endothelium which is essentially similar to that of the peptides they resemble.

For example, compounds like benzolactam or piperazine containing analogues based on the primary sequence of the original peptides can be used (Adams et al., 1999; Nakanishi and Kahn, 1996; Houghten et al., 1999; Nargund et al., 1998). A large variety of types of peptidomimetic substances have been reported in the scientific and patent literature and are well known to those skilled in the art. Peptidomimetic substances (analogues) may comprise for example one or more of the following structural components: reduced amides, hydroxyethylene and/or hydroxyethylamine isosteres, N-methyl amino acids, urea derivatives, thiourea derivatives, cyclic urea and/or thiourea derivatives, poly(ester imide)s, polyesters, esters, guanidine derivatives, cyclic guanidines, imidazoyl compounds, imidazolinyl compounds, imidazolidinyl compounds, lactams, lactones, aromatic rings, bicyclic systems, hydantoins and/or thiohydantoins as well as various other structures. Many types of compounds for the synthesis of peptidomimetic substances are available from a number of commercial sources (e.g. Peptide and Peptidomimetic Synthesis, Reagents for Drug Discovery, Fluka ChemieGmbH, Buchs, Switzerland, 2000 and Novabiochem 2000 Catalog, Calbiochem-Novabiochem AG, Läufelfingen, Switzerland, 2000). The resemblance between the peptidomimetic compounds and the original peptides is based on structural and/or functional similarities. Thus, the peptidomimetic compounds mimic the properties of the original peptides and, for the purpose of the present application, their binding ability is similar to the peptides that they resemble. Peptidomimetic compounds can be made up, for example, of unnatural amino acids (such as D-amino acids or amino acids comprising unnatural side chains, or of b-amino acids etc.), which do not appear in the original peptides, or they can be considered to consist of or can be made from other compounds or structural units. Examples of synthetic peptidomimetic compounds comprise N-alkylamino cyclic urea, thiourea, polyesters, poly(ester imide)s, bicyclic guanidines, hydantoins, thiohydantoins, and imidazol-pyridino-inoles (Houghten et al. 1999 and Nargund et al., 1998). Such peptidomimetic compounds can be characterized as being “structural or functional analogues” of the peptides of this invention.

For the purpose of the present invention, the term “targeting unit” stands for a compound, a peptide or a structural or functional analogue thereof, capable of selectively targeting and selectively binding to tumor tissue, tumors, and, preferably, also to tumor stroma, tumor parenchyma and/or extracellular matrix (ECM) of tumors. More specifically, the targeting units may bind to a cell surface, to a specific molecule or structure on a cell surface or within the cells, or they may associate with the extracellular matrix present between the cells. The targeting units may also bind to the endothelial cells or the extracellular matrix of tumor vasculature. The targeting units may bind also to the tumor mass, tumor cells and extracellular matrix of metastases.

Generally, the.terms “targeting” or “binding” stand for adhesion, attachment, affinity or binding of the targeting units of this invention to tumors, tumor cells and/or tumor tissue to the extent that the binding can be objectively measured and determined e.g., by peptide competition experiments in vivo or ex vivo, on tumor biopsies in vitro or by immunological stainings in situ, or by other methods known by those skilled in the art. Tumor targeting means that the targeting units specifically bind to tumors when administered to a human or animal body. Another term used in the art for this specific association is “homing”. Targeting units and targeting agents according to the present invention are considered to be “bound” to the tumor target in vitro, when the binding is strong enough to withstand normal sample treatment, such as washes and rinses with physiological saline or other physiologically acceptable salt or buffer solutions at physiological pH, or when bound to a tumor target in vivo long enough for the effector unit to exhibit its function on the target.

The binding of the present targeting agents or targeting units, to tumors is “selective” meaning that they do not bind to normal cells and organs, or bind to such to a significantly lower degree as compared to tumors.

Pharmaceutically or physiologically or diagnostically acceptable salts and derivatives of the targeting units and agents of the present invention include salts, esters, amides, hydrazides, N-substituted amides, N-substituted hydrazides, hydroxamic acid derivatives, decarboxylated and N-substituted derivatives thereof. Other suitable pharmaceutically acceptable derivatives are readily acknowledged by those skilled in the art.

The present invention is based on the finding that a group of linear or cyclic peptides having specific amino acid sequences or motifs are capable of selectively targeting tumors, especially NSCLC tumors, in vivo and tumor cells in vitro. Thus, the peptides of this invention, when administered to a human or animal subject, are capable of selectively binding to tumors but do not bind to normal tissue in the body.

The tumor targeting units according to the present invention were identified by bio-panning of phage display libraries. Phage display is a method whereby libraries of random peptides are expressed on the surface of a bacteriophage as part of the phage capsid protein pIII by insertion of its encoding DNA sequence into gene III of the phage genome. The pIII libraries display 3-5 copies of each individual peptide per phage particle (Smith and Scott, 1993).

Phage display peptide libraries were screened by bio-panning to select peptides that are specific to non-small cell lung cancer. The principle of bio-panning comprises 1) exposing homogenized tissue samples to a phage library, 2) washing off unbound phages, and 3) rescuing the phages bound to the target tissue. Repeating steps 1-3 results in a selection of highly enriched peptides having a high binding affinity towards the target tissue compared to other peptides of the original phage library. In the present invention a phage display peptide library was panned against tissue samples taken from primary tumors of non-small cell lung cancer patients, as described in more detail in the Examples-section.

### Targeting Motifs According to the Present Invention

It has now surprisingly been found that a four-amino-acid motif X—R—Y—P, wherein X is alanine, serine or homoserine, or a structural or functional analogue thereof; R is arginine or homoarginine, or a structural or functional analogue thereof; Y is arginine, homoarginine, alanine, leucine, serine, homoserine, valine or proline, or a structural or functional analogue thereof; P is proline, or a structural or functional analogue thereof; targets and exhibits selective binding to tumors and tumor cells and, especially, to NSCLC tumors.

Especially preferred motifs according to the present invention are motifs wherein X is alanine and Y is arginine, i.e., A—R—R—P.

Preferably X is alanine, or a structural or functional analogue thereof, either having no side chain or comprising in its side chain(s) maximally four, more preferably maximally three, still more preferably maximally two, non-hydrogen atoms. Structural or functional analogues of alanine include for example any optical isomers of compounds such as: 3-chloroalanine, 3-fluoroalanine, 2-aminobutanoic acid, 4-fluoro-2-aminobutanoic acid, 4-chloro-2-aminobutanoic acid, 3-cyanoalanine, 3-cyclopropylalanine, 2-amino-3-butenoic acid and 2-amino-3-butynoic acid.

In another preferred embodiment according to the present invention, X is serine or homoserine or a structural or functional analogue thereof, comprising at least one hydroxyl group or other oxygen-containing group capable of hydrogen bond formation, preferably a hydroxyl group.

A structural or functional analogue of serine or homoserine may also be, for example, a homolog thereof; or an amino acid, amino alcohol, diamino alcohol, tri-, oligo- or polyamino alcohol, or amino acid analogue or derivative, that comprises at least one hydroxyl group, esterified hydroxyl grou,p, methoxyl group, other etherified hydroxyl (ether) group, ketoxime group, aldoxime group, hydroxamic acid group, or ketone or aldehyde carbonyl.

Examples of structural or functional analogues of serine or homoserine are any optical isomers of, isoserine, allo-threonine, phenylisoserine, 2-amino-3-(3,4,-dihydroxyphenyl)-3-hydroxypropionic acid, S-(2-hydroxyethyl)-cysteine, 2-amino-4-hydroxypentanedioic acid, O-phospho-serine, O-sulfoserine, statine, beta-(2-thienyl)serine, O-phosphothreonine, 2-amino-3-methoxypropionic acid, as well as thyronine, 4-methoxy-phenylalanine, 2-aminotyrosine, 3-aminotyrosine, 3-iodotyrosine, 3,5-dibromotyrosine, 3,5-diiodotyrosine, any other mono- or di- or tri- or tetrahalogenated tyrosine, 3-nitrotyrosine, 3,5-dinitrotyrosine, O-phosphotyrosine, O-sulfotyrosine, and also compounds such as 2-aminomalonic acid, 2-aminomalonic acid monoethyl ester and 2-amino-3-oxobutanoic acid and its monoesters.

According to the present invention, R includes any optical isomers of arginine, homoarginine and canavanine; and structural or functional analogues thereof preferably comprising at least one guanyl group, amidino group, or related group that has a delocalized positive charge or may obtain it through protonation.

Examples of structural or functional analogues of arginine or homoarginine include: canavanine, 2-amino-8-guanidino-octanoic acid, 2-amino-7-guanidino-octanoic acid, 2-amino-6-guanidino-octanoic acid, 2-amino-5-guanidino-octanoic acid, 2-amino-7-guanidino-heptanoic acid, 2-amino-6-guanidino-heptanoic acid, 2-amino-5-guanidino-heptanoic acid, 2-amino-4-guanidino-heptanoic acid, 2-amino-5-guanidino-hexanoic acid, 2-amino-4-guanidino-hexanoic acid, 2-amino-3-guanidino-hexanoic acid, 2-amino-4-guanidino-pentanoic acid and 2-amino-3-guanidino-pentanoic acid and N-methylated and dimethylated derivatives of these compounds.

According to the present invention Y may be selected from the group consisting of arginine, alanine, leucine, serine, valine or proline, or structural or functional analogues thereof.

Examples of structural or functional analogues of arginine, alanine and serine are described above.

Examples of structural or functional analogues of leucine and valine comprise

(a) amino acids and amino acid analogues and derivatives (such as aminoalcohols and polyamino acids) that comprise as their side chain or side chains or in their side chain or side chains at least one branched, non-branched or alicyclic structure with at least one, preferably at least two similar or different atoms selected from the group consisting of carbon atoms, silicon atoms, halogen atoms bonded to at least one carbon, ether oxygens and thioether sulphurs; or
(b) a branched, non-branched or cyclic non-aromatic, lipophilic or hydrophobic amino acid or amino acid analogue or derivative or a structural or functional analogue thereof, or an amino acid or carboxylic acid or amino acid analogue or derivative or carboxylic acid analogue or derivative that has one or more lipophilic carborane type or other lipophilic boron-containing side chain(s) or its/their equivalent(s) or another lipophilic cage-type structure.

Y can thus be, for example, any optical or geometrical isomer of valine, alanine, isoleucine, leucine, norleucine, norvaline, allo-isoleucine, 2-aminobutanoic acid, 2-amino-2-methylpropionic acid, 2-amino-4,4-dimethylpentanoic acid, 4,5-dehydroleucine, 2-amino-6-isopropylamino-hexanoic acid, 4-amino-6-methylheptanoic acid, 3-amino-6-methylheptanoic acid, 2-amino-6-methylheptanoic acid, tert-leucine, 4-amino-5-cyclohexyl-3-hydroxypentanoic acid, 4-amino-5-cyclohexyl-pentanoic acid, 2-amino-2-cyclohexylacetic acid, 2-amino-3-cyclohexylpropionic acid, 2-amino-4-cyclohexylbutanoic acid, 2-amino-3-cyclopentylpropionic acid, 2-amino-4-cyclopentylbutanoic acid 2-amino-3-cyclobutylpropionic acid, 2-amino-4-cyclobutylbutanoic acid, 2-amino-3-cyclopropylpropionic acid, 2-amino-4-cyclopropylbutanoic acid, 2-amino-3-(1-cyclopentenyl)-propionic acid, 2-amino-4-(1-cyclopentenyl)-butanoic acid, 2-amino-3-ethylsulfanylpropionic acid, 2-amino-3-methylsulfanylpropionic acid, 3-fluoroalanine, 3-chloroalanine, 3,3-dicyclohexylalanine, 2-amino-3-propenoic acid, 2-amino-4,4-dimethylpentanoic acid or statine, or an N-methyl analogue of any one of the aforementioned, an N-ethyl analogue of any of the aforementioned, another N-alkyl analogue of any of the aforementioned, an alphamethyl analogue (2-methyl-analogue) of any of the aforementioned, an alphaethyl analogue (2-ethyl analogue) of any of the aforementioned, or another alpha-alkyl analogue (2-alkyl analogue) of any of the aforementioned; or 2-aminobutanoic acid, 2-amino-2-methylpropionic acid, 4-amino-5-cyclohexyl-3-hydroxypentanoic acid, 4-amino-5-cyclohexylpentanoic acid, 2-amino-2-cyclohexylacetic acid, 2-amino-3-cyclohexylpropionic acid, 2-amino-4-cyclohexylbutanoic acid, 2-amino-3-ethylsulfanylpropionic acid, 2-amino-3-methylsulfanylpropionic acid, 2-amino-4,4,-dimethylpentanoic acid, allo-isoleucine, 4,5-dehydroleucine, 2-amino-6-isopropylamino-hexanoic, norleucine, norvaline, statine, 4-amino-6-methylheptanoic acid, 3-amino-6-methylheptanoic acid, 2-amino-6-methylheptanoic acid or N-methyl analogue of any of the aforementioned, or an N-ethyl analogue, other N-alkyl analogue, alpha-methyl analogue (2-methyl-analogue), alpha-ethyl analogue (2-ethyl analogue) or other alpha-alkyl analogue (2-alkyl analogue) of any of the aforementioned.

According to the present invention, R and Y may also form together a unit comprising any optical isomer of arginine or homoarginine, or an analogue thereof comprising at least one guanyl group, amidino group or related group that has a delocalized positive charge or can obtain it through protonation.

According to the present invention P, includes, any optical or geometrical isomer of proline; as well as structural or functional analogues thereof, comprising a heterocyclic or carbocyclic ring structure, or a structure comprising a double bond; wherein the analogue preferably has steric or nick-forming properties similar or analogous to those of proline.

The motif X—R—Y—P according to the present invention may form part of a larger structure, such as a peptide or some other structure. The compound or structure in question may also comprise more than one motif X—R—Y—P, and the orientation and direction of the motifs may vary.

### Targeting Units According to the Present Invention

It has also been found that peptides, including structural or functional analogues thereof as defined herein, comprising a tumor targeting motif according to the present invention target to and exhibit selective binding to tumors, especially to lung tumors and to non-small cell lung cancer cell tumors. Peptides comprising a tumor targeting motif according to the present invention and, up to seven additional amino acid residues or analogues thereof, likewise exhibit such targeting and selective binding and are especially preferred embodiments of the present invention.

Such peptides are highly advantageous for use as targeting units according to the present invention, e.g., because of their small size and their easy, reliable and cheap synthesis. Due to the small size of the peptides according to the present invention, the purification, analysis and quality control is easy and commercially useful.

Preferred tumor targeting units according to the present invention comprise a tumor targeting motif X—R—Y—P as defined above, and additional residues selected from the group consisting of natural amino acids; unnatural amino acids; amino acid analogues comprising maximally 30 non-hydrogen atoms and an unlimited number of hydrogen atoms; and other structural units and residues whose molecular weight and/or formula weight is maximally 270; wherein the number of said additional residues ranges from 0 to 7, preferably 0 to 6, preferably 0 to 5, preferably 0 to 4 and most preferably 0 to 3.

The targeting units according to the present invention are preferably linear. Linear peptides according to the present invention are fast, easy and cheap to prepare, as they do not require any further processing (cyclization etc.) after synthesis and complicated orthogonal and other protections and extra functional groups are not needed that would be needed for cyclization. It is furthermore easier to link additional units to linear peptides, for example because, there is no need to “reserve” functional groups for the purpose of cyclization, or to use expensive and complicated orthogonal protections, etc. In some preferred embodiments of the present invention, the efficient degradation of linear peptides in the human body is an advantage compared to the use of more slowly degrading substances, e.g., in diagnostic applications where rapid clearance is desired.

In another embodiment of the present invention cyclic peptides may be preferred. Thus the targeting units according to the present invention may also be cyclic. Cyclic peptides are usually more stable in vivo and in many other biological systems than are their non-cyclic counterparts, as is known in the art. More stable peptides according to the present invention are highly preferred for certain purposes, for example in certain therapeutic applications.

Preferred targeting units according to the present invention may comprise a sequence

X—R—Y—P—Zₙ

wherein, X—R—Y—P is a tumor targeting motif as defined above, Z is an amino acid residue or a structural or functional analogue thereof and n is an integer between 0 and 7, preferably 0-6, 0-5, 0-4 and most preferably 0-3.

Especially preferred targeting units are such, where Z is any amino acid residue, except histidine or tryptophane. Especially preferred are targeting units wherein Zₙ comprises at least one of the following: lysine, leucine or aspartic acid, or structural or functional analogues thereof.

Examples of structural or functional analogues of lysine include any optical isomers of lysine or ornithine, and structural and/or functional analogues thereof, that preferably comprise at least one amino group or substituted amino group or other nitrogen-containing group that has or can through protonation gain a positive charge.

Examples of structural or functional analogues of aspartic acid include any optical isomers of glutamic acid or aspartic acid, and structural or functional analogues thereof comprising at least one oxygen atom capable of hydrogen bond formation, and preferably comprising at least one carboxyl group, esterified carboxyl group, hydroxamic acid function, esterified hydroxamic acid function, alcoholic or phenolic hydroxyl group, esterified alcoholic or phenolic hydroxyl group, keto group or aldehyde function, and more preferably comprising at least one carboxyl group; esterified carboxyl group, hydroxamic acid function, esterified hydroxamic acid function, alcoholic or phenolic hydroxyl group or esterified alcoholic or phenolic hydroxyl group, still more preferably comprising at least one carboxyl group, esterified carboxyl group, hydroxamic acid function, alcoholic hydroxyl group or esterified alcoholic hydroxyl group, and most preferably comprising at least one carboxyl group or esterified carboxyl group; or comprising one or more other oxo acid functional groups, selected preferably from the group of: —SO₃⁻, —OSO₃⁻, any inorganic phosphate group or its ester.

Preferred targeting units according to the present invention include those selected from the group consisting of the peptides identified by SEQ ID NO. 1 to SEQ ID NO. 73. Highly preferred targeting units according to the present invention include ARRPKLD (SEQ ID NO. 1), SRRPKLD (SEQ ID NO. 65), ARRP (SEQ ID NO. 66), SRAP (SEQ ID NO. 67), ARAP (SEQ ID NO. 68), SRVP (SEQ ID NO. 69), SRLP (SEQ ID NO. 70), ARLP (SEQ ID NO. 71), ARPP (SEQ ID 72), SRRP (SEQ ID NO. 73).

### Targeting Agents According to the Present Invention

It has now also been found that targeting agents comprising at least one tumor targeting unit according to the present invention, and at least one effector unit, target to and exhibit selective binding to cancer cells and cancer tissues.

The tumor targeting agents according to the present invention may optionally comprise unit(s) such as linkers, solubility modifiers, stabilizers, charge modifiers, spacers, lysis or reaction or reactivity modifiers, internalizing units or internalization enhancers or membrane interaction units or other local route, attachment, binding and distribution affecting units. Such additional units of the tumor targeting agents according to the present invention may be coupled to each other by any means suitable for that purpose.

Many possibilities are known to those skilled in the art for linking structures, molecules and groups of the types in question or of related types, to each other. The various units may be linked either directly or with the aid of one or more identical, similar and/or different linker units. The tumor targeting agents of the invention may have different structures such as any of the non-limiting types schematically shown below:

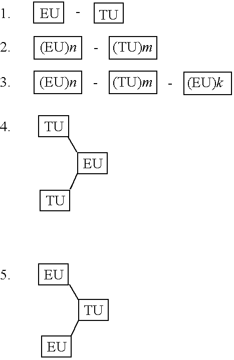

where EU indicates “effector unit” and TU indicates “targeting unit” and n, m and k are independently any integers except 0.

In a targeting agent according to the present invention, as in many other medicinal and other substances, it may be wise to include spacers or linkers, such as amino acids and their analogues, such as long-chain omega-amino acids, to prevent the targeting units from being ‘disturbed’ sterically or electronically, or otherwise hindered or ‘hidden’, by effector units or other unit of the targeting agent.

In targeting agents according to the present invention, it may be useful for increased activity to use dendrimeric or cyclic structures for example to provide a possibility to incorporate multiple effector units or additional units per targeting unit.

Preferred targeting agents according to the present invention comprise a structure EU-TU-OU, TU-EU-OU or TU-OU-EU, wherein TU is a targeting unit according to the present invention as defined above; and EU and OU are effector or optional units selected from the group consisting of:

effector units, linker units, solubility modifier units, stabilizer units, charge modifier units, spacer units, lysis and/or reaction and/or reactivity modifier units, internalizing and/or internalization enhancer and/or membrane interaction units and/or other local route and/or local attachment/local binding and/or distribution affecting units, adsorption enhancer units, and other related units; and

peptide sequences and other structures comprising at least one such unit; and

peptide sequences comprising no more than 20, preferably no more than 12, more preferably no more than 6, natural and/or unnatural amino acids; and

natural and unnatural amino acids comprising no more than 25 non-hydrogen atoms and an unlimited number of hydrogen atoms;

as well as salts, esters, derivatives and analogues thereof.

### Effector Units

For the purposes of this invention, the term “effector unit” (EU) means molecules or radicals or other chemical entities or large particles such as colloidal particles and their like; liposomes, nanoparticles or microgranules. Suitable effector units may also comprise nanodevices or nanochips or their like; or a combination of any of the aforementioned, and optionally chemical structures for the attachment of the constituents of the effector unit to each other or to other parts of the targeting agents. Effector units may also contain moieties that modify the stability or solubility of the effector units.

Preferred effects provided by the effector units according to the present invention are therapeutic (biological, chemical or physical) effects on the targeted tumor; properties that enable the detection or imaging of tumors or tumor cells for diagnostic purposes; or binding abilities that relate to the use of the targeting agents in different applications.

A preferred (biological) activity of the effector units according to the present invention is a therapeutic effect. Examples of such therapeutic activities, are for example, cytotoxicity, cytostatic effects, ability to cause differentiation of cells or to increase their degree of differentiation or to cause phenotypic changes or metabolic changes, chemotactic activities, immunomodulating activities, pain relieving activities, radioactivity, ability to affect the cell cycle, ability to cause apoptosis, hormonal activities, enzymatic activities, ability to transfect cells, gene transferring activities, ability to mediate “knock-out” of one or more genes, ability to cause gene replacements or “knock-in”, ability to decrease, inhibit or block gene or protein expression, antiangiogenic activities, ability to collect heat or other energy from external radiation or electric or magnetic fields, ability to affect transcription, translation or replication of the cell's genetic information or external related information, and to affect post-transcriptional or post-translational events, and so on.

Other preferred therapeutic approaches: enabled by the effector units according to the present invention may be based on the use of thermal (slow) neutrons (to make suitable nuclei radioactive by neutron capture), or the administration of an enzyme capable of hydrolyzing for example an ester bond or other bonds or the administration of a targeted enzyme according to the present invention.

Examples of preferred functions of the effector units according to the present invention suitable for detection are radioactivity, paramagnetism, ferromagnetism, ferrimagnetism, or any type of magnetism, or ability to be detected by NMR spectroscopy, or ability to be detected by EPR (ESR) spectroscopy, or suitability for PET and/or SPECT imaging, or the presence of an immunogenic structure, or the presence of an antibody or antibody fragment or antibody-type structure, or the presence of a gold particle, or the presence of biotin or avidin or other protein, and/or luminescent and/or fluorescent and/or phosphorescent activity or the ability to enhance detection of tumors, tumor cells, endothelial cells and metastases in electron microscopy, light microscopy (UV and/or visible light), infrared microscopy, atomic force microscopy or tunneling microscopy, and so on.

Preferred binding abilities of an effector unit according to the present invention include, for example:

a) ability to bind metal ion(s) e.g. by chelation,
b) ability to bind a cytotoxic, apoptotic or metobolism affecting substance or a substance capable of being converted in situ into such a substance,
c) ability to bind to a substance or structure such as a histidine tag or other tag,
d) ability to bind to an enzyme or a modified enzyme,
e) ability to bind to biotin or analogues thereof,
f) ability to bind to avidin or analogues thereof,
g) ability to bind to integrins or other substances involved in cell adhesion, migration, or intracellular signalling,
h) ability to bind to phages,
i) ability to bind to lymphocytes or other blood cells,
j) ability to bind to any preselected material by virtue of the presence of antibodies or structures selected by biopanning or by other methods,
k) ability to bind to material used for signal production or amplification,
l) ability to bind to therapeutic substances.

Such binding may be the result of e.g. chelation, formation of covalent bonds, antibody-antigen-type affinity, ion pair or ion associate formation, specific interactions of the avidin-biotin-type, or the result of any type or mode of binding or affinity.

One or more of the effector units or parts of them may also be a part of the targeting units themselves. Thus, the effector unit may for example be one or more atoms or nuclei of the targeting unit, such as radioactive atoms or atoms that can be made radioactive, or paramagnetic atoms or atoms that are easily detected by MRI or NMR spectroscopy (such as carbon-13). Further examples are, for example, boron-comprising structures such as carborane-type lipophilic side chains.

The effector units may be linked to the targeting units by any type of bond or structure or any combinations of them that are strong enough so that most, or preferably all or essentially all of the effector units of the targeting agents remain linked to the targeting units during the essential (necessary) targeting process, e.g. in a human or animal subject or in a biological sample under study or treatment.

The effector units or parts of them may remain linked to the targeting units, or they may be partly or completely hydrolyzed or otherwise disintegrated from the latter, either by a spontaneous chemical reaction or equilibrium or by a spontaneous enzymatic process or other biological process, or as a result of an intentional operation or procedure such as the administration of hydrolytic enzymes or other chemical substances. It is also possible that the enzymatic process or other reaction is caused or enhanced by the administration of a targeted substance such as an enzyme in accordance with the present invention.

One possibility is that the effector units or parts thereof are hydrolyzed from the targeting agent or hydrolyzed into smaller units by the effect of one or more of the various hydrolytic enzymes present in tumors (e.g., intracellularly, in the cell membrane or in the extracellular matrix) or in their near vicinity.

Taking into account that the targeting according to the present invention may be very rapid, even non-specific hydrolysis that occurs everywhere in the body may be acceptable and usable for hydrolysing one or more effector unit(s) intentionally, since such hydrolysis may in suitable cases (e.g., steric hindrance, or even without any such hindering effects) be so slow that the targeting agents are safely targeted in spite of the presence of hydrolytic enzymes of the body, as those skilled in the art very well understand. The formation of insoluble products or products rapidly absorbed into cells or bound to their surfaces after hydrolysis may also be beneficial for the targeted effector units or their fragments etc. to remain in the tumors or their closest vicinity.

In one preferred embodiment of the invention, the effector units may comprise structures, features, fragments, molecules or the like that make possible, cause directly or indirectly, an “amplification” of the therapeutic or other effect, of signal detection, of the binding of preselected substances, including biological material, molecules, ions, microbes or cells.

Such “amplification” may, for example, be based on one or more of the following non-limiting types:
the binding, by the effector units, of other materials that can further bind other substances (for example, antibodies, fluorescent antibodies, other “labelled” substances, substances such as avidin), preferably so that several molecules or “units” of the further materials can be bound per each effector unit;
the effector units comprise more than one entity capable of binding e.g. a protein, thus making direct amplification possible;
amplification in more than one steps.

Preferred effector units according to the present invention may be selected from the following group:
cytostatic or cytotoxic agents
apoptosis causing or enhancing agents
enzymes or enzyme inhibitors
antimetabolites
agents capable of disturbing membrane functions
radioactive or paramagnetic substances
substances comprising one or more metal ions
substances comprising boron, gadolinium, litium
substances suitable for neutron capture therapy, e.g. boron or carborane
labelled substances
intercalators and substances comprising them
oxidants or reducing agents
nucleotides and their analogues
metal chelates or chelating agents.

In a highly preferred embodiment of the invention, the effector unit comprises alpha emittors.

In further preferred embodiments of the invention, the effector units may comprise copper chelates such as trans-bis(salicylaldoximato) copper(II) and its analogues, or platinum compounds such as cisplatin and carboplatin.

More specifically, for the treatment of advanced NSCLC in combination with platinum compounds the following agents or their structural or functional analogs may be used: mitosis inhibitors/taxanes such as paclitaxel or docetaxel, or anti-metabolites such as gemsitabine or metotrexate, or vinca alkaloids such as vinorelbine or vincristine, or alkylating agents such as isophosphamide or cyclophosphamide, or antibiotics such as bleomycine or mitomycine, or topoisomerase inhibitors such as irinotecane or topotecane.

Different types of structures, substances and groups are known that can be used to cause or enhance e.g., internalization into cells, including for example RQIKIWFQNRRMKWKK; Penetratin (Prochiantz, 1996), as well as stearyl derivatives (Promega Notes Magazine, 2000).

As an apoptosis-inducing structure, for example, the peptide sequence KLAKLAK that has been reported to interact with mitochondrial membranes inside cells, can be included (Ellerby et al. 1999).

For use in embodiments of the present invention that include cell sorting or any related applications, the targeting units and agents of the invention can, for example, be used

a) coupled or connected to magnetic particles,

b) adsorbed, coupled, linked or connected to plastic, glass or other solid, porous, fibrous material-type or other surface(s) and their like,

c) adsorbed, covalently bonded or otherwise linked, coupled or connected into or onto one or more substance(s) or material(s) that can be used in columns or related systems

d) adsorbed, covalently bonded or otherwise linked, coupled or connected into or onto one or more substance(s) or material(s) that can be precipitated, centrifuged or otherwise separated or removed.

### Optional Units (OU) of the Targeting Agents According to the Present Invention

The targeting agents and targeting units of the present invention may optionally comprise further units, such as:

linker units for coupling targeting units, effector units, or other optional units of the present invention to each other;

solubility modifying units for modifying the solubility of the targeting agents or their hydrolysis products;

stabilizer units for stabilizing the structure of the targeting units or agents during synthesis, modification, processing, storage or use in vivo or in vitro; charge modifying units for modifying the electrical charges of the targeting units or agents or their starting materials;

spacer units for increasing the distance between specific units of the targeting agents or their starting materials, for releasing or decreasing steric hindrance or structural strain of the products or their starting materials;

reactivity modifyer units;

internalizing units or enhancer units for enhancing targeting or up-take of the targeting agents;

adsorption enhancer units, such as fat soluble or water soluble structures that for example enhance absorption of the targeting agents in vivo; or

other related units.

A large number of suitable linker units are known in the art. Examples of suitable linkers are:
1. for linking units that comprise amino groups: cyclic anhydrides, dicarboxylic or multivalent, optinally activated or derivatized, carboxylic acids, compounds with two or more reactive halogens or compounds with at least one reactive halogen atom and at least one carboxyl group;
2. for linking units that comprise carboxyl groups or derivatives thereof: com-pounds with at least two similar or different groups such as amino, substituted amino, hydroxyl, —NHNH₂ or substituted forms thereof, other known groups for the purpose (activators may be used);
3. for linking an amino group and a carboxyl group: for example amino acids or their activated or protected forms or derivatives;
4. for linking a formyl group or a keto group to another group: a compound comprising e.g. at least one —N—NH₂ or —O—NH₂ or ═N—NH₂ group or their like;
5. for linking several amino-comprising units: polycarboxylic substances such as EDTA, DTPA or polycarboxylic acids, or anhydrides, esters or acyl halides thereof;
6. for linking a substance comprising an amino group to a substance comprising either a formyl group or a carboxyl group: hydrazinocarboxylic acids or their like, preferably so that the hydrazino moiety or the carboxyl group is protected or activated, such as 4-(FMOC-hydrazino)benzoic acid;
7. for linking an organic structure to a metal ion: substances that can be coupled to the organic structure (e.g. by virtue of their COOH groups or their NH₂ groups) or that are integral parts of it, and that in addition comprise a polycarboxylic part, for example an EDTA- or DTPA-like structure, peptides comprising several histidines or their like, peptides comprising several cysteines or other moieties comprising an —SH group each, or other chelating agents that comprise functional groups that can be used to link them to the organic structure.

A large variety of the above substances and of other types of suitable linking agents are known in the art.

A large number of suitable solubility modifier units are known in the art. Suitable solubility modifier units may comprise, for example:
for increasing aqeous solubility: molecules comprising SO₃⁻, O—SO₃⁻, COOH, COO—, NH₂, NH₃⁺, OH, phosphate groups, guanidino or amidino groups, or other ionic or ionizable groups or sugar-type structures;
for increasing fat solubility or solubility in organic solvents: units comprising (long) aliphatic branched or non-branched alkyl or alkenyl groups, cyclic non-aromatic groups such as the cyclohexyl group, aromatic rings or steroidal structures.

A large number of units known in the art can be used as stabilizer units, e.g. bulky structures (such as tert-butyl groups, naphthyl and adamantyl and related radicals etc.) for increasing steric hindrance, and D-amino acids and other unnatural amino acids (including beta-amino acids, omega-amino acids, amino acids with very large side chains etc.) for preventing or hindering enzymatic hydrolysis.

Units comprising positive, negative or both types of charges can be used as charge modifier units, as can also structures that are converted or can be converted into units with positive, negative or both types of charges.

Spacer units may be very important, and the need to use such units depends on the other components of the structure (e.g. the type of biologically active agents used, and their mechanisms of action) and the synthetic procedures used.

Suitable spacer units may include for example long aliphatic chains or sugar-type structures (to avoid too high lipophilicity), or large rings. Suitable compounds are available in the art. One preferred group of spacer units are omega-amino acids with long chains. Such compounds can also be used (simultaneously) as linker units between an amino-comprising unit and a carboxyl-comprising unit. Many such compounds are commercially available, both as such and in the forms of various protected derivatives.

Units that are susceptible to hydrolysis (either spontaneous chemical hydrolysis or enzymatic hydrolysis by the body's own enzymes or enzymes administered to the patient) may be very advantageous in cases where it is desired that the effector units are liberated from the targeting agents e.g. for internalization, intra- or extracellular DNA or receptor binding. Suitable units for this purpose include, for example, structures comprising one or more ester or acetal functionality. Various proteases may be used for the purposes mentioned. Many groups used for making pro-drugs may be suitable for the purpose of increasing or causing hydrolysis, lytic reactions or other decomposition processes.

The effector units, the targeting units and the optional units according to the present invention may simultaneously serve more than one function. Thus, for example, a targeting unit may simultaneously be an effector unit or comprise several effector units; a spacer unit may simultaneously be a linker unit or a charge modifier unit or both; a stabilizer unit may be an effector unit with properties different from those of another effector unit, and so on. An effector unit may, for example, have several similar or even completely different functions.

In one preferred embodiment of the invention, the tumor targeting agents comprise more than one different effector units. In that case, the effector units may be, for example, diagnostic and therapeutic units. Thus, for example, it is preferred to use, for boron neutron capture therapy, such agents whose effector units, in addition to comprising boron atoms, also can be detected or quantified in the patient in vivo after administration of the agent, in order to be able to ascertain that the agent has accumulated adequately in the tumor to be treated, or to optimize the timing of the neutron treatment, and so on. This goal may be achieved e.g. by using such a targeting agents according to the invention that comprise an effector unit comprising boron atoms (preferably isotope-enriched boron) and groups detectable e.g. by NMRI. Likewise, the presence of more than one type of therapeutically useful effector units may also be preferred. In addition, the targeting units and targeting agents may, if desired, be used in combination with one or more “classical” or other tumor therapeutic modalities such as surgery, chemotherapy, other targeting modalities, radiotherapy, immunotherapy etc.

### Preparation of Targeting Units and Agents According to the Present Invention

The targeting units according to the present invention are preferably synthetic peptides. Peptides can be synthesized by a large variety of well-known techniques, such as solid-phase methods (FMOC-, BOC-, and other protection schemes, various resin types), solution methods (FMOC, BOC and other variants) and combinations of these. Even automated apparatuses/devices for the purpose are available commercially, as are also routine synthesis and purification services. All of these approaches are very well known to those skilled in the art. Some methods and materials are described, for example, in the following references:

Bachem AG, SASRINä (1999), The BACHEM Practise of SPPS (2000), Bachem 2001 catalogue (2001), Novabiochem 2000 Catalog (2000), Peptide and Peptidomimetic Synthesis (2000) and The Combinatorial Chemistry Catalog & Solid Phase Organic Chemistry (SPOC) Handbook 98/99. Peptide synthesis is exemplified also in the Examples.

As known in the art, it is often advisable, important and/or necessary to use one or more protecting groups, a large variety of which are known in the art, such as FMOC, BOC, and trityl groups and other protecting groups mentioned in the Examples. Protecting groups are often used for protecting amino, carboxyl, hydroxyl, guanyl and —SH groups, and for any reactive groups/functions.

As those skilled in the art well know, activation often involves carboxyl function activation and/or activation of amino groups.

Protection may also be orthogonal and/or semi/quasi/pseudo-orthogonal. Protecting and activating groups, substances and their uses are exemplified in the Examples and are described in the references cited herein, and are also described in a large number of books and other sources of information commonly known in the art (e.g. Protective Groups in Organic Synthesis, 1999).

Resins for solid-phase synthesis are also well known in the art, and are described in the Examples and in the above-cited references.

Cyclic peptides are usually especially stable in biological milieu, and are thus preferred. Cyclic structures according to the present invention may be synthesized by methods based on the use of orthogonally protected amino acids, as described in e.g., International Patent Publication WO 2004/031219, incorporated herein by reference.

The targeting units and agents according to the present invention may also be prepared as fusion proteins or by other suitable recombinant DNA methods known in the art. Such an approach for preparing the peptides according to the present invention is preferred especially when the effector units and/or other optional units are peptides or proteins. One example of a useful protein effector unit is glutathion-S-transferase (GST).

### Advantages of the Targeting Units and Targeting Agents of the Invention

There are acknowledged problems related to peptides intended for diagnostic or therapeutic use. One of these problems stems from the length of the sequence: the longer it grows, the more difficult the synthesis of the desired product becomes, especially if there are other synthetic problems such as the presence of difficult residues that require protection-deprotection or cause side reactions.

As compared to known peptides that contain long and difficult-to-make sequences with problematic amino acid residues, the peptides of the present invention are clearly superior. The targeting units of this invention can be synthesized easily and reliably. An advantage as compared to many prior art peptides is that the targeting units and motifs of this invention do not need to comprise the problematic basic amino acids lysine and histidine, nor tryptophan, all of which may cause serious side-reactions in peptide synthesis, and, due to which the yield of the desired product might be lowered radically or even the product might be impossible to obtain in adequate amounts or with adequate quality.

Because of their smaller size and thus drastically less steps in the synthesis, the peptides of the present invention are much easier and cheaper to produce than most targeting peptides of the prior art.

As histidine is not needed in the products of the present invention, the risk of racemization is of no concern. It is a great advantage not only for the economic synthesis of the products of the present invention but also for the purification and analysis and quality control that any racemization of histidine is outside consideration. It also makes any administration to humans and animals safer and more straightforward.

Because of the smaller size of the targeting units, overall costs are drastically reduced and better products can be obtained and in greater amounts, due to easier and more reliable purification. Furthermore, the reliability of the purification is much better, giving less concern of toxic remainders and of fatal or otherwise serious side-effects in therapeutic and diagnostic applications.

The targeting units of the present invention are also highly advantageous due to their high solubility, specificity, non-toxicity and non-immunogenicity. A great problem of prior art targeting peptides is that their aqueous solubility, or solubility in general, is usually very low or even extremely low. Thus there is an urgent need of targeting peptides having good targeting properties and excellent solubility properties, which are easy to synthesize and purify. This invention provides a solution to this great problem by providing targeting peptides with superior targeting properties, easy and cheap synthesis and purification, and with extremely good solubility in water, even coupled to carboranes that are extremely hydrophobic.

In the solid phase synthesis of targeting agents according to the present invention, the effector units and optional additional units may be linked to the targeting peptide when it is still connected to the resin, without the risk that the removal of the protecting groups will cause destruction of the effector or optional units. Similar advantages apply to solution syntheses.

Another important advantage of the present invention and the products, methods and uses according to it is the highly selective and potent targeting of the products.

As compared to targeted therapy using antibodies or antibody fragments, the products and methods of in the present invention are highly advantageous because of several reasons. Potential immunological and related risks are obvious in the case of large biomolecules; Allergic reactions are of great concern with such products; in contrats to small synthetic molecules such as the targeting agents, units and motifs of the present invention.

As compared to targeting antibodies or antibody fragments, the products and methods described in the present invention are highly advantageous because their structure can be easily modified if needed or desired. Specific amino acids such as histidine, tryptophan, tyrosine and threonine can be omitted, if desired, and very few functional groups are necessary. On the other hand, it is possible, without disturbing the targeting effect, to include various different structural units, to obtain specific desired properties that are of special value in specific applications.

### Use of the Targeting Agents According to the Present Invention

The targeting units and targeting agents according to the present invention are useful in cancer diagnostics and therapy, as they selectively target to tumors, especially to NSCLC tumors in vivo, as shown in the Examples. The effector unit may be chosen according to the desired effect, detection or therapy. The desired effect may also be achieved by including the effector in the targeting unit as such. For use in radiotherapy the targeting unit itself may be e.g., radioactively labelled.

The present invention also relates to diagnostic compositions comprising an effective amount of at least one targeting agent according to the present invention. A diagnostically effective amount of the targeting agents according to the present invention may range from 1 femtomol to 10 mmols, depending for example on the effector unit of choice. In addition to the targeting agent, a diagnostic composition according to the present invention may, optionally, comprise carriers, solvents, vehicles, suspending agents, labeling agents and other additives commonly used in diagnostic compositions. Such diagnostic compositions are useful in diagnosing tumors, tumor cells and metastasis, especially tumors of the lung, more specifically non-small cell lung cancer tumors and adenocarcinomas of the NSCLC type.

A diagnostic composition according to the present invention may be formulated as a liquid, gel or solid formulation or as an inhalation formulation, etc., preferably as an aqueous liquid, containing a targeting agent according to the present invention in a concentration ranging from about 1×10⁻¹⁰ mg/l to 25×10⁴ mg/l. The compositions may further comprise stabilizing agents, detergents, such as polysorbates, as well as other additives. The concentrations of these components may vary significantly depending on the formulation used. The diagnostic compositions may be used in vivo or in vitro.

The present invention also includes the use of the targeting agents and targeting units for the manufacture of pharmaceutical compositions for the treatment of cancer.

The present invention also relates to pharmaceutical compositions comprising a therapeutically effective amount of at least one targeting agent according to the present invention. The pharmaceutical compositions may be used to treat, prevent or ameliorate cancer diseases, by administering a therapeutically effective dose of the pharmaceutical composition comprising targeting agents or targeting units according to the present invention or therapeutically acceptable salts, esters or other derivatives thereof. The compositions may also include different combinations of targeting agents and targeting units together with labelling agents, imaging agents, drugs and other additives.

A therapeutically effective amount of a targeting agent according to the present invention may vary depending on the formulation of the pharmaceutical composition. Preferably, a pharmaceutical composition according to the present invention may comprise a targeting agent in a concentration varying from about 0.00001 mg/l to 250 g/l, more preferably about 0.001 mg/l to 50 g/l, most preferably 0.01 mg/I to 20 g/l.

A pharmaceutical composition according to the present invention is useful for administration of a targeting agent according to the present invention. Pharmaceutical compositions suitable for peroral use, for intravenous or local injection, or infusion, or inhalation are particularly preferred. The pharmaceutical compositions may be used in vivo or ex vivo.

The preparations may be lyophilized and reconstituted before administration or may be stored for example as a solutions, suspensions, suspension-solutions etc. ready for administration or in any form or shape in general, including powders, concentrates, frozen liquids, and any other types. They may also consist of separate entities to be mixed and, possibly, otherwise handled and/or treated etc. before use. Liquid formulations provide the advantage that they can be administered without reconstitution. The pH of the solution product is in the range of about 1 to about 12, preferably close to physiological pH. The osmolality of the solution can be adjusted to a preferred value using for example sodium chloride and/or sugars, polyols and/or amino acids and/or similar components. The compositions may further comprise pharmaceutically acceptable excipients and/or stabilizers, such as albumin, sugars and various polyols, as well as any acceptable additives, or other active ingredients such as chemotherapeutic agents.

The present invention also relates to methods for treating cancer, especially solid tumors by administering to a patient in need of such treatment a therapeutically efficient amount of a pharmaceutical composition according to the present invention.

Therapeutic doses may be determined empirically by testing the targeting agents and targeting units in available in vitro or in vivo test systems. Suitable therapeutically effective dosage may then be estimated from these experiments.

For oral administration it is important that the targeting units and targeting agents are stable and adequately absorbed from the intestinal tract.

The pharmaceutical compositions according to the present invention may be administered systemically, non-systemically, locally or topically, parenterally as well as non-parenterally, e.g. subcutaneously, intravenously, intramuscularly, perorally, intranasally, by pulmonary aerosol or powder, by injection or in-fusion into a specific organ or region, buccally, intracranically or intraperitoneally etc.

Amounts and regimens for the administration of the tumor targeting agents according to the present invention can be determined readily by those with ordinary skill in the clinical art of treating cancer. Generally, the dosage will vary depending upon considerations such as: type of targeting agent employed; age; health; medical conditions being treated; kind of concurrent treatment, if any; frequency of treatment and the nature of the effect desired; gender; duration of the symptoms; and, counterindications, if any, and other variables to be adjusted by the individual physician. Preferred doses for administration to human patients of targeting units or agents according to the present invention may vary from about 1×10⁻⁹ mg to about 40 mg per kg of body weight as a bolus or repeatedly, e.g., as daily doses.

The targeting units and targeting agents and pharmaceutical compositions of the present invention may also be used as targeting devices for delivery of DNA or RNA or structural and functional analogues thereof, such as phosphorothioates, or peptide nucleic acids (PNA) into tumors and their metastases or to isolated cells and organs in vitro; i.e. as tools for gene therapy both in vivo and in vitro. In such cases the targeting agents or targeting units may be parts of viral capsids or envelopes, of liposomes or other “containers” of DNA/RNA or related substances, or may be directly coupled to the DNA/RNA or other molecules mentioned above. An especially preferred embodiment of the present invention is a targeting agent comprising a TU as an amino acid chain or its structural or functional analogue, and an EU as a PNA or its analogue, linked together via a peptide bond, as one contiguous molecule. Such a targeting agent may be used for intracellular delivery of small interfering RNA (siRNA; in this case “siPNA”) for gene product-specific inhibition (silencing) of gene expression.

The present invention also includes kits and components of kits for diagnosing, detecting or analysing cancer or cancer cells in vivo and in vitro. Such kits comprise at least one targeting agent or targeting unit of this invention together with diagnostic entities enabling detection. The kit may comprise for example a targeting agent or a targeting unit coupled to a unit for detection by e.g. immunological methods, radiation or enzymatic methods or other methods known in the art.

Further, the targeting units and agents of this invention as well as the targeting motifs and sequences can be used as lead compounds to design peptidomimetics for any of the purposes described above.

Yet further, the targeting units and agents as well as the targeting motifs and sequences of the present invention, as such or as coupled to other materials, can be used for the isolation, purification and identification of the cells, molecules and related biological targets.

The following examples are given to further illustrate preferred embodiments of the present invention, but are not intended to limit the scope of the invention. It will be obvious to a person skilled in the art, as the technology advances, that the inventive concept can be implemented in various ways. The invention and its embodiments are not limited to the examples described above but may vary within the scope of the claims.

### EXAMPLES

### Example 1

### General Screening Method for Bio-Panning of Patient Samples

Phage display libraries. Standard procedures according to Smith and Scott (1993) were used. Phage display libraries used for screening of clinical samples were cloned in fUSE5 vectors and were of the structure X7 and X10, thus they were linear containing seven or ten random amino acids. The libraries were used separately or as a mixture. The E. coli strain K91kan was used as host for phage amplification.

Subtractional panning. Bio-panning was started with depletion of phage clones binding to normal lung. Normal lung tissue taken from surgical lung resection, removed during dissection of tumor, was placed in ice cold DMEM (Dulbecco's medium) containing protease inhibitors (PI); 10 mM PMSF (Para-methyl-sulphonyl-fluoride), Aprotinin (10 mg/ml) Leupeptin (10 mg/ml). Tissue samples were minced with a razor blade in a small cell culture plate in 1 ml of DMEM-PI. The samples were transferred to an eppendorf tube and washed with 1 ml DMEM-PI.

Samples were centrifuged at 4000 rpm for 5 min and were then incubated with 10¹⁰ transforming units (TU) of phage (from one or more peptide libraries) in 1 ml DMEM-PI at 25° C. for 45 min. After this the samples were washed three times with DMEM-PI containing 1% BSA (bovine serum albumin).

1 ml K91kan bacteria, OD600 (optical density of 600 nm) 1-1.5, in LB (Lurian broth) containing 100 μg/ml kanamycin (kan) were infected with the supernatant containing phage particles not binding to normal lung tissue at 25° C. for 25 min. After infection volume was increased to 2 ml with LB containing 100 μg/ml kan. Then infected bacteria were plated on LB agar plates containing 40 μg/ml tetracycline (tet) in 200 μl aliquots. The plates were incubated overnight at +37° C.

The next day the bacterial colonies were pooled together from the plates in 200 ml LB (100 μg/ml kan, 20 μg/ml tet) for further growth. The culture was grown at 37° C. for 1-1.5 h.

Then the bacteria were pelleted at 5000 rpm for 15 min. The supernatant containing amplified phages was precipitated by adding PEG (polyethyleneglycol) to 0.04 g/ml and NaCl to 0.03 g/ml. The phages were shaken overnight at +4° C. on ice. After this the phages were pelleted by centrifugation at 10 000 rpm for 20 min at +4° C. The resulting pellet was re-suspended in TBS (Tris-buffer saline) and then re-precipitated for 1 h at +4° C. on ice by addition of PEG/NaCl as described above.

Then the phages were pelleted at 14 000 rpm for 20 min at +4° C. on ice. Finally, the pellet was re-suspended in 1 ml of TBS containing 0.02% NaN₃ and stored at +4° C.

Titration of phage. For the next rounds of bio-panning of clinical samples the titer of the TBS phage stock was determined as follows: Several dilutions (1:1000-1:1×10⁷) were done for infection of the host bacteria. After infection, bacteria were plated on LB agar plates containing 40 μg/ml tetracycline (tet) and the plates were incubated overnight at +37° C.

The following day the titer was calculated by counting colonies (TU/ml TBS phage stock).

Phage display on clinical tumor samples. Tissue samples were surgically removed from primary tumors of non-small cell lung cancer patients and placed in ice DMEM-PI. Part of sample was taken for pathological examination. The type and nature of the tumor samples were first verified as being NSCLC. After that, specification of subtype of NSCLC and its stage was done by pathologists.

Tissue samples were minced with a razor blade in a small cell culture plate in 1 ml of DMEM containing protease inhibitors. The samples were transferred to an eppendorf tube and washed with 1 ml DMEM-PI.

Samples were centrifuged at 4000 rpm for 5 min and were then incubated with 10¹⁰ TU of phage (from one or more peptide libraries) in iml DMEM-PI at 25° C. for 15 min. After this the samples were washed three times with DMEM-PI containing 1% BSA (bovine serum albumin).

1 ml K91kan bacteria, OD600: 1-1.5, in LB containing 100 μg/ml kanamycin (kan) were infected with the washed pellet containing selectively attached phage particles at 25° C. for 25 min. After infection volume was increased to 2 ml with LB containing 100 μg/ml kan.

Then infected bacteria were plated on LB agar plates containing 40 μg/ml tetracycline (tet) as follows: Two parallel plates of three dilutions (1:50, 1:500, 1:5000) and the rest of the above K91kan culture in 200 μl aliquots. The plates were incubated overnight at +37° C.

The following day 24-48 colonies were picked from the LBtet plates into 96-well micro-plates for sequencing of the phage DNA. Alternatively the clones were stored for later analysis at −20° C.

After picking colonies for sequencing the remaining bacterial colonies were pooled from the plates in 200 ml LB (100 μg/ml kan, 20 μg/ml tet) for further growth. The culture was grown at 37° C. for 1-1.5 h.

Then the bacteria were pelleted at 5000 rpm for 15 min. The supernatant containing amplified phages was precipitated by adding PEG to 0.04 g/ml and NaCl to 0.03 g/ml. The phages were shaken overnight at +4° C. on ice. After this the phages were pelleted by centrifugation at 10 000 rpm for 20 min at +4° C. The resulting pellet was re-suspended in TBS and then reprecipitated for 1 h at +4° C. on ice by addition of PEG/NaCl as described above. Then the phages were pelleted at 14 000 rpm for 20 min at +4° C. on ice. Finally, the pellet was re-suspended in 1 ml of TBS containing 0.02% NaN₃ and stored at +4° C. For the next rounds of bio-panning of clinical samples the titre of the TBS phage stock was determined as described above.

To achieve selective enrichment of tumor targeting peptides, phage stocks prepared as described above were used three to six rounds of biopanning of clinical samples.

Monitoring of enrichment of peptides. To determine the number of sequence of selectively enriched peptides, DNA sequencing was performed on 24 to 48 colonies, representing individual phage clones, from the second round of bio-panning onwards.

First colony PCR was performed to produce DNA for sequencing: Bacterial colonies in the wells of 96-well plate were suspended to 10 ml TBS buffer and 5 μl of this were taken to PCR reaction. Next, PCR-Mix was made—PCR-Mix for one reaction is: 0.1 ml 10 mM dNTP's, 5.0 μl of template, 0.7 μl of F1-forward primer (15 μM), 0.7 μl F1-reverse primer (15 μM), 4 μl 10×Dynazyme buffer, 0.5 μl of Dynazyme polymerase (=1U) and 29 μl of dH2O giving a final volume of 40 ml. The setting for the PCR program used was 96° C. for 5 min followed by a cycle of three steps 1) 92° C. for 30 seconds, 2) 60° C. for 30 seconds and 3) 72° C. for 1 minute. This cycle of three steps was repeated 35 times. The sequences of the primers used in PCR amplification were 5′-gCMgCTgATAAACCgATACMTTAAAgg-3′ for F1-F and 5′-gCCC TCA TAg TTA gCg TM CgA TC-3′ for F1-R.

Prior to sequencing amplification of DNA insert of the phage clones was verified by electrophoresis. Sequencing was performed with an ALF automated DNA sequencer (AmershamPharmacia Biotech) using the F1-F and F1-R primers described above.

Peptide sequences selectively enriched by bio-panning of human lung tumor tissue. Peptides selectively binding to lung tumors are listed in Table 1. The enriched peptide sequences were collected from ex vivo panning rounds four to six.

TABLE 1

| | |
|---|---|
| Sequence | Frequency |

| | |
|---|---|
| ARRPKLD | 22 |
| ARPPKGVNWT | 3 |
| ARLPQVELSA | 3 |
| ARAPGVMPTT | 2 |
| ARMPPRS | 2 |
| ARRPATL | |
| ARRPAVAAFE | |
| ARRPPQM | |
| ARQPAHF | |
| ARKPVFQ | |
| ARNPTLGNSS | |
| ARPPRST | |
| ARSPRVK | |
| ARSPHVTPIA | |
| ARSPISP | |
| ARYPVTM | |
| ARTPSRTPVV | |
| ARAPKMG | |
| ARAPGVR | |
| ARAPGPPRLA | |
| ARAPKMG | |
| ARAPYAS | |
| ARMPQYT | |
| ARLPRAVVPL | |
| SRNPGLLTVR | 2 |
| SRAPNSVQHD | 2 |
| SRAPVAP | 2 |
| SRLPSAGTFQ | 2 |
| SRRPAIM | |
| SRRPVWF | |
| SRRPQLP | |
| SRRPAFVVRV | |
| SRRPGLSHAA | |
| SRRPLVV | |
| SRQPTSL | |
| SRSPRVVEGL | |
| SRSPLVV | |
| SRYPVVS | |
| SRTPPLL | |
| SRSPRLALPT | |
| SRSPVMS | |
| SRYPLEL | |
| SRWPGSV | |
| SRPPART | |
| SRAPLLRPII | |
| SRAPVAP | |
| SRAPLGSIAD | |
| SRAPAVAGWK | |
| SRAPAQKVFFG | |
| SRAPSNVERM | |
| SRAPSTLAHV | |
| SRAPSPSYRQ | |
| SRMPGSV | |
| SRMPLPV | |
| SRMPTLMSGL | |
| SRLPEVVLGQ | |
| SRLPART | |
| SRLPVSATLA | |
| SRVPGRATAT | |
| SRVPLGP | |
| SRVPLGRASS | |
| SRVPSDV | |
| SRVPYQN | |
| SRVPVRGVFQ | |

### Example 2

### Preparation of Synthetic Peptides

All peptide syntheses were carried out manually or by means of an automated synthesis instrument (either Applied Biosystems 433A or Advanced Chem Tech 396DC). The method was solid phase peptide synthesis based on N-FMOC protection and HBTU/HOBt/DIPEA activation. The synthesis resins employed were Rink amide MBHA resin, cysteamine-2-chlorotrityl resin or pre-loaded FMOC-amino acid Wang resin. In automated syntheses the standard operating procedures and reagents recommended by the manufacturers were employed.

The major reagents in these syntheses were from Applied Biosystems or from Novabiochem: Fmoc-Ala-OH (for ‘A’), Fmoc-Asp(OtBu)-OH (for ‘D’), Fmoc-Gly-OH (for ‘G’), Fmoc-Lys(tBoc)-OH (for ‘K’), Fmoc-Leu-OH (for ‘L’), Fmoc-Pro-OH (for ‘P’), Fmoc-Arg(Pbf)-OH (for ‘R’). The spacer amino acid: Fmoc-11-amino-3,6,9-undecanoic acid (for ‘PEG’) was purchased, University of Kuopio, Finland, and had been prepared as described previously (Boumrah et al., 1997).

Linkers: 2-Aminoethanethiol was produced via the cleavage of the cysteamine resin. Fmoc-Lys(Mtt)-OH was employed for the production of a branched structure by virtue of the orthogonal protection of the two amino groups. The metal chelating agent Dota, i.e.1,4,7,10-tetraazacyclododecane-1,4,7,10-tetraacetic acid, coupled via one carboxyl, was incorporated by means of solid phase coupling of Dota tris(t-Bu ester) from Macrocyclics, Dallas, Tex.

Labels: The thiol-reactive labelling reagent, the europium(III) chelate of p-iodoacetamidobenzyl-DTPA from Perkin Elmer, was coupled with sulf-hydryl bearing peptide compound according to Perkin Elmer's recommended procedure.

The following abbreviations are used herein:

‘Ac’ denotes: CH₃C(O) i.e. acetyl (not actinium).
‘ADGA’ denotes: Ala-Asp-Gly-Ala.
‘AMB-DTPA-Eu’ denotes:
Eu³⁺-chelate of (p-((2-aminoethylmercapto)acetamido)benzyl)diethylenetriamine-N1, N2, N3, N3-pentaacetic acid coupled via primary amino group (at the aminoethyl group).
‘amide’ denotes: NH₂ group connected to carbonyl (e.g. at the C-terminus of a peptide).
‘Dota’ denotes: 1,4,7,10-tetraazacyclododecane-1,4,7,10-tetraacetic acid coupled via one carboxyl, i.e. (CH₂CH₂N(CH₂COOH))₄ minus one OH.
‘DPLKRAR’ denotes: Asp-Pro-Leu-Lys-Arg-Ala-Arg.
‘DTPA’ denotes: diethylenetriamine-N1, N2, N3, N3-pentaacetic acid.
‘DTPA-Eu’ denotes: Eu³⁺-chelate of DTPA
‘EAT’ denotes: 2-Aminoethanethiol, i.e. ethyleneaminothiol, i.e —NHCH₂CH₂SH.
‘G3’ denotes Gly-Gly-Gly.
‘GA’ denotes: Gly-Ala.
‘GAAG’ denotes: Gly-Ala-Ala-Gly.
‘PEG’ denotes: NH—CH₂CH₂—O—CH₂CH₂—O—CH₂CH₂—O—CH₂—C(O).
‘EG’ denotes: NH—CH₂CH₂—O—CH₂CH₂—O—CH₂CH₂NH.
‘Biotin’ denotes: D-biotinyl, i.e. vitamin H coupled via its carboxyl group.
‘Carborane’ denotes: 5-(1-o-carboranyl)-pentanoyl moiety, C(O)—CH₂)₄—C₂B₁₀H₁₁.

### LIST OF REAGENTS

Fmoc-Gly-OH, CAS No. 29022-11-5, Novabiochem Cat. No. 04-12-1001 Molecular Weight: 297.3 g/mol.
Fmoc-L-Arg(Pbf)-OH, CAS No. 154445-77-9, Applied Biosystems Cat. No. GEN911097, Molecular Weight: 648.8 g/mol.
Fmoc-L-Leu-OH, CAS No. 35661-60-0, Applied Biosystems Cat. No. GEN911048, MolecularWeight: 353.4 g/mol.
Fmoc-L-Pro-OH, CAS No. 71989-31-6, Applied Biosystems Cat. No. GEN911060, Molecular Weight: 337.4 g/mol.
Fmoc-L-Lys-OH, CAS No. 71989-26-9, Applied Biosystems Cat. No. GEN911051, Molecular Weight: 468.6 g/mol.
Cysteamine-2-chlorotrityl Resin, Novabiochem 01-64-0107, subst.: 1.33 mmol/g.
Rink amide MBHA Resin, Novabiochem 01-64-0107, subst.: 1.33 mmol/g.
Fmoc-Gly Resin, Applied Biosystems Cat. No. 401421, 0.65 mmol/g.
Fmoc-Gly Resin (for carboxy-terminal ‘Gly-OH’), Applied Biosystems Cat. No. 401421, 0.65 mmol/g.
O-bis-(aminoethyl)ethylene glycol trityl resin (for ‘EG’), Novabiochem product No. 01-64-0235.
D-Biotin (Vitamin H), CAS No. 58-85-5, Sigma B-4501, molecular weight: 244.3 g/mol.
5-(1-o-carboranyl)-pentanoic acid (for ‘carborane’), Katchem, Prague, Czech Republic, molecular weight: 244.34 g/mol.
General Procedures For Peptide Synthesis: Manual Solid Phase Syntheses. Mass Spectral Measurements.

All manual synthetic procedures were carried out in a sealable glass funnel equipped with a sintered glass filter disc of porosity grade between 2 and 4, a polypropene or phenolic plastic screw cap on top (for sealing), and two PTFE key stopcocks: one beneath the filter disc (for draining) and one at sloping angle on the shoulder of the screw-capped neck (for argon gas inlet).

The funnel was loaded with the appropriate solid phase synthesis resin and solutions for each treatment, shaken effectively with the aid of a “wrist movement” bottle shaker for an appropriate period of time, followed by filtration effected with a moderate argon gas pressure.

The general procedure of one cycle of synthesis (=the addition of one amino acid unit) was as follows:

The appropriate synthesis resin (from Applied Biosystems or Novabiochem), loaded with approximately 0.25 mmol of FMOC-peptide (=peptide whose amino-terminal amino group was protected with the 9-fluorenylmethyloxycarbonyl group) consisting of one or more amino acid units having recommended protecting groups; approximately 0.5 g of resin (0.5 mmol/g) was treated in the way described below, each treatment step comprising shaking for one to two minutes with 10 ml of the solution or solvent indicated and filtration if not mentioned otherwise.

‘DCM’ means shaking with dichloromethane, and ‘DMF’ means shaking with N,N-dimethylformamide (DMF may be replaced by NMP, i.e., N-methylpyrrolidinone).

The steps of the treatment were:

1. DCM, shaking for 10-20 min

### 2. DMF

3. 20% (by volume) piperidine in DMF for 5 min
4. 20% (by volume) piperidine in DMF for 10 min

### 5. to 7. DMF

### 8. to 10. DCM

### 11. DMF

12. DMF solution of 0.75 mmol of activated amino acid (preparation described below), shaking for 2 hours

### 13. to 15. DMF

### 16. to 18. DCM

After the last treatment (18) argon gas was led through the resin for approximately 15 min and the resin was stored under argon (in the sealed reaction funnel if the synthesis was to continue with further units).

Activation of the 9-fluorenylmethyloxycarbonyl-N-protected amino acid (FMOC-amino acid) to be added to the amino acid or peptide chain on the resin was carried out, using the reagents listed below, in a separate vessel prior to treatment step no. 12. Thus, the FMOC-amino acid (0.75 mmol) was dissolved in approximately 3 ml of DMF, treated for 1 min with a solution of 0.75 mmol of HBTU dissolved in 1.5 ml of a 0.5 M solution of HOBt in DMF, and then immediately treated with 0.75 ml of a 2.0 M DIPEA solution for 5 min.

The activation reagents used for activation of the FMOC-amino acid were as follows:

HBTU=2-(1H-benzotriazol-1-yl)-1,1,3,3-tetramethyluronium hexafluorophosphate, CAS No. [94790-37-1], Applied Biosystems Cat. No. 401091, molecular weight: 379.3 g/mol
HOBt=1-Hydroxybenzotriazole, 0.5 M solution in DMF, Applied Biosystems Cat. No. 400934
DIPEA=N,N-Diisopropylethylamine, 2.0 M solution in N-methylpyrrolidone, Applied Biosystems Cat. No. 401517

The procedure described above is repeated in several cycles using different FMOC-amino acids, containing suitable protecting groups, to produce a “resin-bound” peptide (i.e., resinous source of an appropriate peptide). The procedure provides also a way to connect certain effector or linker units, for instance Dota or FMOC-Teg (i.e., Fmoc-11-amino-3,6,9-undecanoyl moiety), to the resin-bound peptide. Also the very first unit (at the C-terminal end of the sequence) can be connected to Rink amide resin or to cysteamine resin by means of this general coupling method described above; in the case of cysteamine resin the initial treatment with piperidine (steps 3 to 11) is not necessary at the first cycle.

When N-terminally acetylated product was needed the procedure above was carried out with the exception of acetic anhydride instead of the activated FMOC-amino acid at step 12 using reagent mixture: one volume of acetic anhydride mixed in four volumes of, 2.0 M solution of N,N-diisopropylethylamine in N-methylpyrrolidone.

Cleavage from the resin was carried out using the following reagent mixture:

trifluoroacetic acid (TFA) 92.5 vol-%
water 5.0 vol-%
ethanedithiol 2.5 vol-%.

After the removal of the protecting FMOC group via steps 1. to 10. (as described in the general procedure above), the resin was washed with DCM, dried at argon flow and treated with three portions of the above reagent mixture (each about 10 ml), each for one hour. The treatments were carried out under argon atmosphere in the way described above. After three hours from the beginning of the treatment the TFA solutions obtained by filtration were concentrated under reduced pressure using a rotary evaporator and were recharged with argon. Purification was done by reversed phase high-performance liquid chromatographic (HPLC) methods using a “Waters 600” pump apparatus with a C-18 type column of particle size 10 micrometers, and a linear eluent gradient whose composition was changed during 30 minutes from 99.9% water/0.1% TFA to 99.9% acetonitrile/0.1% TFA. The dimensions of the HPLC columns were 25 cm×21.2 mm (Supelco cat. no. 567212-U) and 15 cm×10 mm (Supelco cat. no. 567208-U). Detection was based on absorbance at 218 nm and was carried out using a “Waters 2487” instrument.

The cleavage mixture described above also simultaneously removed the following protecting groups: Tert-butoxycarbonyl (Boc) as used for protection of side chain of lysine; 2,2,4,6,7-pentamethyldihydrobenzofuran-5-sulfonyl (Pbf) as used for protection of side chain of arginine; tert-buthyl ester (OtBu) as used for protection of side chain carboxyl group of aspartic acid, and can normally be used also for removal of these protecting groups on analogous structures (thiol, guanyl, carboxyl).

The compound synthesized this way is constructed from “right to left” in the conventionally (also in this text) presented sequence, i.e. starting from the C-terminal end of the peptide chain.

### Mass Spectral Method Employed:

### Matrix Assisted Laser Desorption Ionization—Time of Flight (MALDI-TOF)

### Type of the Instrument:

Bruker Ultraflex MALDI TOF/TOF mass spectrometer

### Supplier of the Instrument:

Bruker Daltonik GmbH

Fahrenheitstrasse 4

D-28359 Bremen

Germany

### MALDI-TOF Positive Ion Reflector Mode:

External standards:
Angiotensin II, angiotensin II, substance P (RPKPQQFFGLM), bombesin, ACTH(1-17) ACTH(18-39), somatostatin 28 and bradykinin 1-7.

### Matrix:

alpha-cyano-4-hydroxycinnamic acid (2 mg/mL solution in aqueous 60% acetonitrile containing 0.1% of trifluoroacetic acid, or acetone only for acid sensitive samples).

### MALDI-TOF Negative Ion Reflector Mode:

External standards:
cholecystokinin and glucagon or [Glu1]-fibrinogen peptide B.

### Matrix:

alpha-cyano-4-hydroxycinnamic acid (saturated solution in acetone)

### Sample Preparation:

The specimen was mixed at a 10-100 picomol/microliter concentration with the matrix solution as described and dried onto the target.

Ionization by “shooting” in vacuo by nitrogen laser at wavlength 337 nm. The Voltage of the probe plate was 19 kV in positive ion reflector mode and −19 kV in the negative ion reflector mode.

### General Remarks About the Spectra (Concerning Positive Ion Mode Only):

In all cases the M+1 (i.e. the one proton adduct) signal with its typical fine structure based on isotope satellites was clearly predominant. In almost all cases, the M+1 signal pattern was accompanied by a similar but markedly weaker band of peaks at M+23 (Na+ adduct). In addition to the bands at M+1 and M+23, also bands at M+39 (K+ adduct) or M+56 (Fe+ adduct) could be observed in many cases.

The molecular mass values reported within synthesis examples correspond to the most abundant isotopes of each element, i.e. the ‘exact masses’.

### Example 3

### The Synthesis of Targeting Agent IS257

The synthesis of Ac-ARRPKLD-amide (IS257), i.e. CH₃C(O)-Ala-Arg-Arg-Pro-Lys-Leu-Asp-NH₂, was carried out manually according to the general method described above and was based on Rink amide MBHA Resin. The reagents (as described in the List of Reagents) were used according the sequence above in the direction of the syntesis (starting from Fmoc-Asp(OtBu)-OH, i.e. from right to left).

The identification of the product was based on MALDI-TOF mass spectrum: Observed positive ion M+1: 896.51 Da.

Calculated isotopic M: 895.54 Da.

### Example 4

### The Synthesis of Targeting Agent HP196

The synthesis of ADGA-ARRPKLD-GAAG (HP196), i.e. H-Ala-Asp-Gly-Ala-Ala-Arg-Arg-Pro-Lys-Leu-Asp-Gly-Ala-Ala-Gly-N H₂, was carried out manually according to the general method described above and was based on Rink amide MBHA Resin. The reagents (as described in the list of reagents) were used according the sequence above in the direction of the syntesis (starting from Fmoc-Gly-OH, i.e. from right to left).

The identification of the product was based on MALDI-TOF mass spectrum: Observed positive ion M+1: 1424.8 Da.

Calculated isotopic M: 1423.8 Da.

### Example 5

### The Synthesis of Targeting Agent HP199

The synthesis of a targeting agent ADGA-ARRPKLD-GMG-PEG-G3-EAT comprising the targeting unit ARRPKLD included in peptide sequence ADGA-ARRPKLD-GAAG and also comprising a sulfhydryl bearing linker unit via a spacer units at the C-terminus of the peptide sequence was carried out by means of Applied Biosystems 433A peptide synthesis instrument and bands at M+1 and M+23, also bands at M+39 (K+ adduct) or M+56 (Fe+ adduct) could be observed in many cases.

The molecular mass values reported within synthesis examples correspond to the most abundant isotopes of each element, i.e. the ‘exact masses’.

### Example 3

### The Synthesis of Targeting Agent IS257

The synthesis of Ac-ARRPKLD-amide (IS257), i.e. CH₃C(O)-Ala-Arg-Arg-Pro-Lys-Leu-Asp-NH₂, was carried out manually according to the general method described above and was based on Rink amide MBHA Resin. The reagents (as described in the List of Reagents) were used according the sequence above in the direction of the syntesis (starting from Fmoc-Asp(OtBu)-OH, i.e. from right to left).

The identification of the product was based on MALDI-TOF mass spectrum: Observed positive ion M+1: 896.51 Da.

Calculated isotopic M: 895.54 Da.

### Example 4

### The Synthesis of Targeting Agent HP196

The synthesis of ADGA-ARRPKLD-GAAG (HP196), i.e. H-Ala-Asp-Gly-Ala-Ala-Arg-Arg-Pro-Lys-Leu-Asp-Gly-Ala-Ala-Gly-NH₂, was carried out manually according to the general method described above and was based on Rink amide MBHA Resin. The reagents (as described in the list of reagents) were used according the sequence above in the direction of the syntesis (starting from Fmoc-Gly-OH, i.e. from right to left).

The identification of the product was based on MALDI-TOF mass spectrum: Observed positive ion M+1: 1424.8 Da.

Calculated isotopic M: 1423.8 Da.

### Example 5

### The Synthesis of Targeting Agent HP199

The synthesis of a targeting agent ADGA-ARRPKLD-GAAG-PEG-G3-EAT comprising the targeting unit ARRPKLD included in peptide sequence ADGA-ARRPKLD-GAAG and also comprising a sulfhydryl bearing linker unit via a spacer units at the C-terminus of the peptide sequence was carried out by means of Applied Biosystems 433A peptide synthesis instrument and based on cysteamine-2-chlorotrityl resin and solid phase Fmoc-chemistry and regular protected amino acid reagents (including unusual Fmoc-PEG-OH that was used in the regular manner).

The structure of the targeting agent is: Ala-Asp-Gly-Ala-Ala-Arg-Arg-Pro-Lys-Leu-Asp-Gly-Ala-Ala-Gly-NH—CH2CH₂—O—CH₂CH₂—O—CH₂CH₂—O—CH₂—C(O)-Gly-Gly-Gly-NHCH₂CH₂SH.

The identification of the product was based on MALDI-TOF mass spectrum: Observed positive ion M+1: 1845.27 Da.

Calculated isotopic M: 1843.93 Da.

### Example 6

### The Synthesis of Targeting Agent HP201

The synthesis of targeting agent Ac-ARRPKLD-GAAG-PEG-G3-EAT comprising targeting unit ARRPKLD and sulfhydryl bearing linker agent via spacer units at the C-terminus of the targeting unit was carried out by means of Applied Biosystems 433A peptide synthesis instrument based on cysteamine-2-chlorotrityl resin and solid phase Fmoc-chemistry and regular protected amino acid reagents (including unusual Fmoc-PEG-OH that was used in the regular manner). The arginine next to proline was coupled by double treatment and the N-terminus was capped by acetylation.

The structure of the targeting agent is: Ala-Gly-NH—CH₂CH₂—O—CH₂CH₂—O—CH₂CH₂—O—CH₂—C(O)-Gly3-NHCH₂CH₂SH.

The identification of the product was based on MALDI-TOF mass spectrum: Observed positive ion M+1: 1572.85 Da.

Calculated isotopic M: 1571.82 Da.

### Example 7

### The Synthesis of Targeting Agent A48

The synthesis of targeting agent Ac-ARRPKLD-GA-EAT comprising the targeting unit ARRPKLD included in the peptide sequence ARRPKLD-GA and also comprising a sulfhydryl bearing linker unit at the C-terminus of the targeting unit was carried out by means of Applied Biosystems 433A peptide synthesis instrument based on cysteamine-2-chlorotrityl resin and solid phase Fmoc-chemistry and regular protected amino acid reagents. The N-terminus was acetylated.

The structure of the targeting agent is: CH₃C(O)-Ala-Asp-Gly-Ala-Ala-Arg-Arg-Pro-Lys-Leu-Asp-Gly-Ala-NHCH₂CH₂SH.

The identification of the product was based on MALDI-TOF mass spectrum: Observed positive ion M+1: 1085 Da.

Calculated isotopic M: 1083.60 Da.

### Example 8

### Synthesis of Europium-Labelled Targeting Agent A48-Eu

The targeting agent Ac-ARRPKLD-GA-EAT having a mercapto group at its C-terminal end was treated with thiol reactive (iodoacatamido activated) IAA-DTPA europium chelate from Perkin-Elmer (PerkinElmer Life Sciences and Analytical Sciences—Oy, Turku, Finland) according to Perkin-Elmers's protocol.

Thus 6.6 mg of peptide (code A48) was dissolved in 1 mL of 0.05 M NaHCO₃. The Eu³⁺-chelate of (p-iodoacetamindobenzyl)diethylenetriamine-N1, N2, N3, N3-pentaacetic acid (8 mg) in 1.5 mL of 0.05 NaHCO₃ was added to the peptide solution. After pH was adjusted to 8.5 the solution was protected from light and allowed to stay overnight at 30° C. The product: CH₃C(O)-Ala-Arg-Arg-Pro-Lys-Leu-Asp-Gly-Ala-NHCH₂CH₂S-p-CH₂CONH-benzyl-DTPA europium chelate was purified by RP-HPLC at water-acetonitrile eluent gradient buffered by 0.05 M ammonium acetate, and identified by means of negative-ion mode MALDI-TOFF mass spectrum. Observed negative ion M-1: 1770.74 Da with typical isotopic distribution.

Calculated isotopic M: 1771.68 Da.

### Example 9

### The Synthesis of Targeting Agent A49

The synthesis of targeting agent Ac-ARRPKLD-GAAG-PEGSU-EAT comprising the targeting unit ARRPKLD and also comprising a sulfhydryl bearing linker unit via spacer units at the C-terminus of the targeting unit [‘PEGSU’ denotes: NH—(CH₂)₃—(O—CH₂CH₂)₃—CH₂—NH—C(O)CH₂CH₂C(O)] was carried out by means of Applied Biosystems 433A peptide synthesis instrument based on cysteamine-2-chlorotrityl resin and solid phase Fmoc-chemistry and regular protected amino acid reagents with the exception of the first amino acid: 1-amino-4,7,10-trioxa-13-tridecanamine succinamic acid. The reagent for that was produc No. FA18801 of NeoMPS (Strasbourg, France): Fmoc-1-amino-4,7,10-trioxa-13-tridecanamine succinimic acid, and was used in the synthesis like regular Fmoc-amino acid. The N-terminus was acetylated.

The structure of the targeting agent is: CH₃C(O)-Ala-Arg-Arg-Pro-Lys-Leu-Asp-Gly-Ala-Ala-Gly-NH—(CH₂)₃—(O—CH₂CH₂)₃—CH₂—NH—C(O)CH₂CH₂—C(O)—NHCH₂CH₂SH.

The identification of the product was based on MALDI-TOF mass spectrum: Observed positive ion M+1: 1514.86 Da.

Calculated isotopic M: 1513.84 Da.

### Example 10

### Synthesis of Targeting Agent F5M-A49

Fluorescein labeled targeting agent A49-F was synthesized using fluorescein-5-maleimide (Promega). In this reaction the peptide A49 was coupled to the maleimide part of the label through its sulfhydryl group. In the coupling reaction the F5M and A49 were made to 4 mM in coupling buffer (10 mM Tris/HCl pH 7.5, 5 mM Na₂HPO₄, 2 mM EDTA). The molarity of F5M in the reaction is three times the molarity of A49. The reaction was carried out by mixing at 37° C. overnight protected from light. The reaction was ended with addition of β-mercaptoethanol and the reaction product was purified using HPLC after which it was lyophilised. For use the F5M-A49 was dissolved to PBS pH 7.4.

The structure of the targeting agent is: CH₃C(O)-Ala-Arg-Arg-Pro-Lys-Leu-Asp-Gly-Ala-Ala-Gly-NH—(CH₂)₃—(O—CH₂CH₂)₃—CH₂—NH—C(O)CH₂CH₂—C(O)—NHCH₂CH₂S—C₂H₃(COOH)—C(O)—NH—(C₂₀H₁₁O₅), i.e. fluorescein-5-succinamide acid thioether derivative of A49.

The identification of the product was based on MALDI-TOF mass spectrum: Observed positive ion M+1: 1959.95 Da.

Calculated isotopic M: 1958.92 Da.

### Example 11

### Synthesis of Targeting Agent HP192

The synthesis of a targeting agent Dota-Lys(Ac-ARRPKLD-(PEG)2)-amide (HP192) comprising the targeting unit ARRPKLD and metal chelating agent Dota via spacer units at the C-terminus of the targeting unit was carried out manually, according to the general method described above, and was based on Fmoc-Lys(Mtt)-OH coupled with Rink amide MBHA resin. Dota tris-t-Bu-ester was coupled with Lys(Mtt) on resin in the ordinary way. Before the continuation of the synthesis the protecting 4-methyltrityl group (i.e. Mtt) was cleaved off the side-branch of the lysine moiety by means of two subsequent 10 minutes' treatments with the reagent mixture: 4% trifluoroacetic acid/1% ethanedithiol in dichloromethane. The washings after the deprotection were: twice with dichloromethane, once with 0.1 M ethyl-N,N-diisopropylamine in dichloromethane and three times with N,N-dimethylformamide prior to the coupling of the first Fmoc-PEG-OH. The synthesis continued manually according to the general method employing the appropriate amino acid reagents: Fmoc-PEG-OH (two cycles), Fmoc-Asp(OtBu)-OH, Fmoc-Leu-OH, Fmoc-Lys(tBoc)-OH, Fmoc-Pro-OH, Fmoc-Arg(Pbf)-OH (two cycles), and Fmoc-Ala-OH. The final end capping, for two hours, was carried out with the reagent mixture: one volume of acetic anhydride mixed in four volumes of 2 M ethyl-N,N-diisopropylamine in N-methylpyrrolidinone (i.e. NMP). After washings with three portions of N,N-dimethylformamide and four portions of dichloromethane the product was isolated in the ordinary way desribed above.

The structure of the targeting agent is: Dota-Lys[CH₃C(O)-Ala-Arg-Arg-Pro-Lys-Leu-Asp-PEG-PEG]-NH₂.

The peptide sequence ARRPKLD is acetylated at the N-terminus and is coupled with the side branch of lysine via two spacer amino acid units (PEG). ‘PEG’ denotes: NH—CH₂CH₂—O—CH₂CH₂—O—CH₂CH₂—O—CH₂—C(O). ‘Dota’ denotes: 1,4,7,10-tetraazacyclododecane-1,4,7,10-tetraacetic acid coupled via one carboxyl, i.e. (CH₂CH₂N(CH₂COOH))₄ minus one OH.

The identification of the product was based on MALDI-TOF mass spectrum: Observed positive ion M+1: 1788.94 Da.

Calculated isotopic M: 1788.0 Da.

### Example 12

### The Synthesis of HP186—the Starting Material for Compounds HP187 and IS248

The synthesis of the source material for targeting agent Ac-ARRPKLD-EG-H comprising the targeting unit ARRPKLD, and also comprising a spacer unit at the C-terminus of the peptide sequence was carried out manually according to the general method described above and was based on O-bis-(aminoethyl)ethylene glycol trityl resin from Novabiochem (product No. 01-64-0235). The reagents (as described in the List of Reagents) were used according the sequence above in the direction of the syntesis (starting from Fmoc-Asp(OtBu)-OH, i.e. from right to left) and the N-terminus was capped by acetylation. The cleavage off the resin was carried out in different manner from the general procedure to maintain the protective groups: The resin was treated with two portions of 2% (by volume) trifluoroacetic acid in dichloromethane for 15 minutes each. The filtered solutions were poured on amounts of pyridine equimolar to the acid and the product was precipitated with water and dried in vacuo. The product was used as such and the identification was based on the analysis of the furter products (codes HP186 and IS248).

The structure of the source compound is: CH₃C(O)-Ala-Arg(Pbf)-Arg(Pbf)-Pro-Lys(tBoc)-Leu-Asp(OtBu)-NH(CH₂CH₂O)₂CH₂CH₂N H₂. “EG” denotes: NH(CH₂CH₂O)₂CH₂CH₂NH—.

### Example 13

### The Synthesis of Targeting Agent HP187

The synthesis of targeting agent Ac-ARRPKLD-EG-Biotin (HP187) comprising the targeting unit ARRPKLD, and also comprising biotin bearing linker unit via a spacer unit at the C-terminus of the peptide sequence, was carried out on bis-(6-carboxy-HOBt)-N-(2-aminoethyl)-aminomethyl polystyrene resin from Novabiochem (product No. 01-64-0179). Afer the resin was shaken with a mixture of threefold excess of biotin and PyBroP (Bromo-trispyrrolidinophosphonium hexafluorophosphate, CAS No. 132705-51-2, Molecular weight: 466.2 g/mol, Novabiochem product No. 01-62-0017) and sixfold excess of DIPEA in N,N-dimethylformamide for five hours the, resin was washed with DMF and dichloromethane as described above in the general manual solid phase synthesis method. The treatment with biotin was repeated for 13 hours followed by washings. Next, 25% excess of the resin was shaken overnight with the protected targeting unit comprising source compound described above (code HP186) in N,N-dimethylformamide. The solution was filtered, the residue extracted with dichloromethane, and the combined solutions evaporated to dryness and treated with the 92% TFA/water reagent mixture regularly used for the deprotection (and cleavage off the resin) as described above and purified by HPLC.

The structure of the targeting agent is: CH₃C(O)-Ala-Arg-Arg-Pro-Lys-Leu-Asp-NH(CH₂CH₂O)₂CH₂CH₂NH-Biotinyl, where biotin is coupled via its carboxyl group to the “EG” spacer at the C-terminal end of the targeting unit.

The identification of the product was based on Q-TOF ES+ mass spectrum: Observed positive ion M+1: 1253.81 Da.

Calculated isotopic M: 1252.71 Da.

### Example 14

### The Synthesis of Targeting Agent IS248

The synthesis of targeting agent Ac-ARRPKLD-EG-Carborane (IS248), comprising the targeting unit ARRPKLD, and also comprising multiple boron bearing effector unit via a spacer unit at the C-terminus of the peptide sequence.

The structure of the targeting agent is: CH₃C(O)-Ala-Arg-Arg-Pro-Lys-Leu-Asp-NH(CH₂CH₂O)₂CH₂CH₂NHC(O)—(CH₂)₄-(1-o-carboranyl ), where 5-(1-o-carboranyl)-pentanoic acid is coupled via its carboxyl group to the “EG” spacer at the C-terminal end of the targeting unit.

The synthesis of targeting agent Ac-ARRPKLD-EG-Carborane (IS244), comprising the targeting unit ARRPKLD, and also comprising multiple boron bearing effector unit via a spacer unit at the C-terminus of the peptide sequence was carried out in organic solution. Thus 0.185 mol of 5-(1-o-carboranyl)-pentanoic acid and 0.192 mol of WSC (1-ethyl-3-(3′-dimethyl-aminopropyl)carbodiimide.HCl, CAS No. 25952-53-8, MW.155.2+36.5) from Novabiochem (product No. 01-62-0011) were dissolved in 1.5 mL of dichloromethane. After 20 min 0.062 mol of the protected targeting unit comprising source compound described above (code HP186) was combined in the described solution and stirred overnight at room temperature. Next, the mixture was evaporated to dryness and treated for two hours with 95% TFA/5% water mixture. After evaporation the residue was triturated by diethyl ether and the precipitate was purified by HPLC.

The structure of the targeting agent is: CH₃C(O)-Ala-Arg-Arg-Pro-Lys-Leu-Asp-NH(CH₂CH₂O)₂CH₂CH₂NHC(O)—(CH₂)₄-(1-o-carboranyl), where 5-(1-o-carboranyl)-pentanoic acid is coupled via its carboxyl group to the “EG” spacer at the C-terminal end of the targeting unit.

The identification of the product was based on MALDI-TOF mass spectrum: Observed positive ion M+1: 1254.87 Da for the highest peak of the typical isotopic pattern contributed by 10 boron atoms.

Calculated isotopic M: 1254.86 Da (1253.89 Da for the highest peak of the isotopic pattern).

### Example 15

### Synthesis of the Targeting Unit Variants

Fifteen compounds were synthesised: Six variations of the targeting peptide ARRPKLD (i.e. Ala-Arg-Arg-Pro-Lys-Leu-Asp) by replacement one of the amino acid residues with A (i.e. alanine), i.e. AARPKLD, ARAPKLD, AR-RAKLD, ARRPALD, ARRPKAD, and ARRPKLA. Five variations of the targeting peptide by replacement of K (i.e. lysine) with D (i.e. aspartic acid), O (i.e. ornithine), R (i.e. arginine), or Y (i.e. tyrosine), i.e. ARRPDLD, ARRPOLD, ARRPRLD, or ARRPYLD. Four variations of the targeting peptide by replacement of L (i.e. leusine) with I (i.e. isoleusine), V (i.e. valine), or F (i.e. phenylalanine), i.e. ARRPKID, ARRPKVD, and ARRPKFD. Finally two variations of the targeting peptide by replacement of D (i.e. aspartic acid) with N (i.e. asparagine) or K (i.e. lysine), i.e. ARRPKLN and ARRPKLK.

The fifteen syntheses were carried out by means of Advanced Chem Tech 396DC multi-channel peptide synthesis instrument (Supplier: Advanced Chemtech, Louisville, Ky., USA) and were based on preloaded Wang resins. The synthetic method was solid phase peptide synthesis based on N-FMOC protection and HBTU/HOBt/DIPEA activation. The standard operating procedures and reagents recommended by the manufacturer of the instrument were employed.

### Example 16

### Synthesis of Control Peptide BTK148

The synthesis of a comparison peptide ADGA-DPLKRAR-GAAG was carried out by means of Advanced Chem Tech 396DC peptide synthesis instrument and based on glycine Wang resin and solid phase Fmoc-chemistry and regular protected amino acid reagents.

The structure: H-Ala-Asp-Gly-Ala-asp-Pro-Leu-Lys-Arg-Ala-Arg-Gly-Ala-Ala-Gly-OH.

The identification of the product was based on MALDI-TOF mass spectrum: Observed positive ion M+1: 1424.75 Da.

Calculated isotopic M: 1425.8 Da.

### Example 17

### Synthesis of Control Peptide HP205

The synthesis of comparison compound ADGA-DPLKRAR-GAAG-PEG-G3-EAT with scrambled peptide sequence and sulfhydryl bearing linker unit via spacer units at the C-terminus of the peptide sequence was carried out by means of Applied Biosystems 433A peptide synthesis instrument and was based on cysteamine-2-chlorotrityl resin and solid phase Fmoc-chemistry and regular protected amino acid reagents (including unusual Fmoc-PEG-OH that was used in the regular manner).

The structure of the compound is: H-Ala-Asp-Gly-Ala-asp-Pro-Leu-Lys-Arg-Ala-Arg-Gly-Ala-Ala-Gly-NH-CH₂CH₂—O—CH₂CH₂—O—CH₂CH₂—O—CH₂—C(O)-Gly3-NHCH₂CH₂SH.

The identification of the product was based on MALDI-TOF mass spectrum: Observed positive ion M+1: 1844.91 Da.

Calculated isotopic M: 1843.93 Da.

### Example 18

### Description of Cell Lines Used in vitro and in vivo Tests

In the examples the following cell lines and culture conditions were used, where not otherwise indicated:

The non-small cell lung cancer (NSCLC) adenocarcinoma cell line NCI-H23, called herein also “NCI-H23”, has been described previously (Little et al., 1983). The cell line was cultured in RPMI 1640 medium with 2 mM L-glutamine adjusted to contain 1.5 g/L sodium bicarbonate, 4.5 g/L glucose, 10 mM HEPES, and 1.0 mM sodium pyruvate, 1% penicillin/streptomycin, 10% fetal bovine serum.

The NSCLC adenocarcinoma cell line A549, called herein also “A549”, has been described previously (Giard et al., 1973). Ham's F-12 medium adjusted to contain 2 mM L-glutamine, 1% penicillin/streptomycin, and 10% fetal bovine serum.

The NSCLC epidermoid carcinoma cell line NCI-H520, called herein also “NCI-H520”, has been described previously (Banks-Schlegel et al., 1985). The cell line was cultured in RPMI 1640 medium with 2 mM L-glutamine adjusted to contain 1.5 g/L sodium bicarbonate, 4.5 g/L glucose, 10 mM HEPES, and 1.0 mM sodium pyruvate, 1% penicillin/streptomycin, 10% fetal bovine serum.

The NSCLC large cell carcinoma cell line NCI-H460, called herein also “NCI-H460”, has been described previously (Banks-Schlegel et al., 1985). The cell line was cultured in RPMI 1640 medium with 2 mM L-glutamine adjusted to contain 1.5 g/L sodium bicarbonate, 4.5 g/L glucose, 10 mM HEPES, and 1.0 mM sodium pyruvate, 1% penicillin/streptomycin, 10% fetal bovine serum.

The human primary pulmonary artery smooth muscle cells (PASMC), called herein also “PASMC” (CAMBREX, CC-2581) were cultured using Clonetics SmGM®-2 BulletKit (CC-3182). The Intraepithelial carcinoma cell line HeLa, called herein also “HeLa”, has been described previously (Scherer et al., 1953). The cell line was cultured in Dulbecco's Modified Eagle Medium (DMEM) medium adjusted to contain 2 mM L-glutamine, 1% penicillin/streptomycin, and 10% fetal bovine serum.

The mouse fibroblast line NIH3T3, called herein also “NIH3T3”, has been described previously (Koga et al., |1979). The cell line was cultured in DMEM medium adjusted to contain 2 mM L-glutamine, 1% penicillin/streptomycin, and 10% fetal bovine serum.

The mouse embryo endothelial cell line E10V, called herein also “E10V”, has been described previously (Garlanda et al., 1994). The cell line was cultured in DMEM medium adjusted to contain 2 mM L-glutamine, 1% penicillin/streptomycin, and 10% fetal bovine serum.

The mouse vascular endothelial cell line SVEC4-10, called herein also “SVEC4-10”, has been described previously (O'Connell, 1990). The cell line was cultured in DMEM medium adjusted to contain 2 mM L-glutamine, 1% penicillin/streptomycin, and 10% fetal bovine serum.

The human melanoma cell line C8161/M1, called herein also “C8161/M1”, has been described previously (Bregman, 1986). The cell line was cultured in DMEM medium adjusted to contain 2 mM L-glutamine, 1% penicillin/streptomycin, and 10% fetal bovine serum.

### Example 19

### Selective Binding of Non-Small Cell Lung Cancer Cells to Immobilized Targeting Agents

Preparation of plates for assays. Wells of Reacti-Bind Maleimide activated clear strip plate (Pierce, Prod#. 15150) were coated with targeting agents of this invention at a concentration of 30 μg/ml. The incubation was carried out of overnight at 20° C. The binding buffer containing unbound peptide was removed from the wells.

The wells were blocked with blocking buffer (1.0% BSA, 0.05% Tween20 in phosphate buffer saline (PBS) pH 7.0. PBS was prepared as follows: 40 g of NaCl, 1 g of KCl, 7 g of Na₂HPO₄×2H₂O and 1 g of KH₂PO₄ were dissolved to 1000 ml of deionized H₂O). Blank wells as controls were prepared by treating empty wells with blocking buffer. The plates were incubated 1 hour 30 min at 20° C.

After incubation the plate was washed three times with PBS. Cell binding assays. 75000 cells in volume of 150 μl of medium were added into coated wells and were incubated for 30 minutes at 37° C. After cell binding, the wells were washed with PBS for 30 minutes. Detection of targeting agent bound cells were based on the MTT assay (described in detail in Example 23, Cytotoxicity). 10 μl of MTT reagent and 90 μl of medium were added to the wells. The plate was incubated for three hours at +37° C. After the incubation, 100 μl of lysis buffer was added to the wells and let to incubated o/n 37° C. On following day the absorbance of plate was measured at 560 nm with ELISA-reader (ThermoLabsystems, multiskan EX).

Cell lines NCI-H23, NCI-H520, A549, HeLa and NIH3T3 (described in Example 18) and targeting agents HP199, HP201 and HP205 (described in Examples 5, 6, 17) were used in the cell binding.

The results of the cell binding assay proving the highly selective binding of NSCLC cell lines to the targeting agentsare shown in FIG. 1. The NSCLC cell lines NCI-H23 (A), A549 (B) and NCI-H520 (C) bind selectively to the immobilized targeting agents HP199 (1) and HP201 (2), whereas the control cell lines PASMC (D), a human primary pulmonary artery smooth muscle cell line, and NIH3T3 (E), a mouse fibroblast cell line do not. Also, the NSCLC cell lines do not bind to the control peptide HP205 (3). The results are shown as measured absorbance at 560 nm.

### Example 20

### Selective Binding of Fluorescent Targeting Agent to Non-Small Cell Lung Cancer Cells

A549 cells and HeLa cells (described in Example 18) were grown on glass slides, washed with PBS and then fixed with methanol. The fluorescent targeting agent F5M-A49 (described in Example 10) was used to stain these cells as follows: Cells were first blocked with blocking buffer (1.0% BSA, 0.05% Tween20 in PBS, pH 7.4) for one hour at 20° C. The cells on the glass slides were incubated with 20 μl of F5M-A49 targeting agent (50 μg/ml in PBS, pH 7.4). As control, binding of F5M-A49 was competed with 20 μl of IS257 targeting unit (free peptide described in Example 3), 500 μg/ml in PBS, pH 7.4) prior to addition of the targeting agent. As negative controls, cells not incubated with any targeting agent were used. After the staining the cells were mounted on object glasses with mountex (Histolab Products Ltd). After this cells were viewed under a fluorescent microscope (Carl Zeiss Microscopy, Jena, Germany). This analysis showed strong staining of A549 cells, no staining of HeLa cells, and F5M-A49 binding to A549 cells was blocked with the free peptide IS257. Thus the staining results prove the selective binding of the fluorescein labelled targeting agent (F5M-A49) to A549 NSCLC cell line.

### Example 21

### Targeting Agent Cell Binding Competition Assay

5000 cells, A549, NCI-H23, NCI-H520, NCI-H460, and control cells PASMC and HeLa (described in Example 18), are grown in multi-well plates, according to the conditions described in Example 18. Targeting agent A48-Eu (described in Example 8) is added to the wells to give a final concentration of 5 pM, and then the cells are incubated for 30 min at 37° C. For the competition assay, 50 pM of the different targeting units, targeting unit variants and the control peptide (described in Examples 3, 4, 15 and 16), each in its own set of wells, are added 15 min prior to addition of A48-Eu targeting agent.

After 30 min of incubation cells are washed 5 times with PBS. PBS is removed and cells are lysed by shaking in Inducer Solution (Perkin-Elmer Ltd) for 15 min. After this, fluorescence is measured by time resolved fluorescence using a Victor III fluorometer (Perkin-Elmer Ltd).

The results show that binding of the targeting agent A48-Eu to NSCLC cells is selectively blocked by all targeting unit variants containing the XRXP motif in their sequence. Furthermore, no binding of targeting agent A48-Eu is observed for the control cell lines PASMC and HeLa.

### Example 22

### In vivo Biodistrution of Targeting Agent in Tumor-Bearing Mice

In this example biodistribution of the targeting agent A48-Eu (described in Example 8) is shown for two different types of primary tumors, A549 and NCI-H520. It is shown that the tested targeting agent according to the present invention selectively targets to primary tumors in vivo but not to normal tissues or organs.

For production of experimental tumors 1×10⁷ cells of A549 and NCI-H520 NSCLC lines (described in Example 18) were injected subcutaneously into both flanks of athymic-nu nude mice strain (Harlan Laboratories). Tumors were harvested when they had reached a weight of about 0.2 g. Tumor-bearing mice were anesthesized by s.c. injection of 60 pl of Domitor (1 mg/ml methyl-parahydroxybenz., 1 mg/ml propyl-parahydroxybenz., 9 mg/ml natrium chloride in 1 ml of sterile water, from Orion Pharma) and 40 μl Ketalar (50 mg/ml ketamin, 0.1 mg/ml benzethon. Chlorid., in 1 ml of sterile water, from Pfizer) prior to administering 0.02 ml/g body weight of Avertin [10 g 2,2,2-tribromoethanol (Fluka) in 10 ml 2-methyl-2-butanol (Sigma Aldrich)] intraperitoneally (i.p.).

To determine the biodistribution pattern of the targeting agent A48-Eu, 275 nmol of A48-Eu targeting agent was injected into the tail vein of athymic nude mice in a volume of 200 μl in physiological saline solution (Baxter). Targeting agent was allowed to circulate for 15 min. Mice were then perfused through the heart with 60 ml of physiological saline. Organs and tissues, including tumors were collected.

For determination of the Eu content of the various tissues, 0.2 g of the tissue samples were taken for analysis using inductively-coupled plasma mass spectrometry (ICP-MS). The samples were dissolved in a microwave oven in a mixture of HNO₃—H₂O₂ (2.5 ml HNO₃+0.5 ml H₂O₂). The samples were then diluted to 30 ml using 1% HNO₃. 10 ng/ml beryllium was then added to the samples as internal standard. The whole samples were then analyzed using standard ICP-MS equipment (VG Plasma Quad. 2+; Varian). The results were calculated as ng lanthanide per g of mouse tissue (Table 2).

TABLE 2

| Tissue | Eu content as (ng/g) |
|---|---|
| Tumour (A549) | 225 +/− 28.6 |
| Tumour (NCI-H520) | 101 +/− 18.1 |
| Muscle | 14.3 +/− 4.77 |
| Brain | 11.3 +/− 0.25 |
| Heart | 4.59 +/− 1.38 |
| Ovaries | 30.2 +/− 12.7 |
| Lungs | 11.5 +/− 4.80 |
| Intestine | 20.7 +/− 4.72 |
| Spleen | 27.7 +/− 22.9 |
| Kidney | 1072 +/− 295 |
| Liver | 144 +/− 33.8 |

The comparison of the amount of europium detected in the mouse tissues showed that the A48-Eu targeting agent accumulated strongly and selectively in A549 tumors as compared to normal tissue, except for the kidney and liver showing high signal due to excretion of the agent via these routes.

The observed high tumor-to-muscle ratio of 15.7:1, further proves the highly selective binding of A48-Eu to A549 tumors. Also NCI-H520 tumors showed significantly selective accumulation of the targeting agent.

Thus, the used targeting agent shows highly selective tumor targeting properties.

### Example 23

### Cytotoxicity Assay

In this assay cell lines were exposed to two different concentrations (50 μg/and 500 μg/ml) of IS257 targeting unit (described in Example 3) for two to three days to test the toxicity of the peptides. The measurement of cell viability was done with MTT (Thiazolyl blue, Sigma M-5655 ) tetrazolium salt. MTT is cleaved to water-insoluble formazan dye by the “succinate-tetrazolium reductase” system which is active only in viable cells. After formazan was solubilized by 10% SDS-0.01 M HCl, it was quantified in an ELISA spectrometer (ThermoLabsystems Multiscan EX) at 560 nm. CuSAO₂ [trans-bis(salicylaldoximato) copper(II)] (Elo HO, Lumme PO., 1985) 7.5 μg/ml was used as a positive control for 100% toxicity.

Procedure. Cells were trypsinized from the cell culture dish (ø9 cm) with 1 ml of TE for 1-5 minutes and moved to a 50 ml Falcon tube. After this the volume was increased to 20 ml of cell line specific medium and cells were transferred to a Bürker chamber and diluted in medium to a concentration of 2500-3500 cells/100 μl depending on cell line. Two or three 96-well micro plates, 24 h, 48 h (and 72 h) were prepared as follows: the first column of the 96-well plate was filled with 100 μl medium/well (w/o cells), and the rest of the columns needed for the experiment with 100 μl of the cell solution so that each well contains 2500-3000 cells. After this the cells were let to attach over night in a cell culture incubator.

Next day 40 μl of medium was removed from all wells except from the ones with only medium and one column with cells (if different cell lines were used in the same plate a one column of each cell line was left untouched).

Then 40 μl of IS257 targeting unit in appropriate medium were added to the wells in two concentrations, so that final concentrations were 50 μg/ml and 500 μg/ml, and the volume of the wells was raised back to 100 μl. Similarly, 40 μl of reference substance Cu(SAO)₂ were added to all the wells in one column so that the final concentration was 7.5 μg/ml. The plates were incubated in an incubator for 24 h (48 h or 72 h). The next day the cell morphology was analyzed with a microscope. After this 10 μl of MTT reagent 5 mg/ml in PBS were added to all wells on the plate and the plate was incubated for 3 h at 37° C. Finally, 100 μl of 10% SDS in 0.01M HCl were added to all the wells and the microplate was incubated over night at 37° C.

The next day, the MTT assay described above was performed. The viable count (v.c.) was calculated as:

Average toxicated cell absorbance−Average DMEM absorbance=Viable count Average living cell absorbance−Average DMEM absorbance

Cell lines tested. Altogether nine cell lines, all described in Example 18, were tested against IS257 targeting unit:

| NSCLC cell lines | | Other cell lines | |
|---|---|---|---|
| A549 | adenocarcinoma | C8161/M1 | melanoma |
| NCI-H23 | adenocarcinoma | HeLa | intraepithelial carcinoma |
| NCI-H520 | epithelial carcinoma | NIH3T3 | mouse fibroblast |
| NCI-H460 | large cell carcinoma | E10V | mouse embryo endothelium |
| | | SVEC4-10 | mouse vascular |
| | | | endothelium |

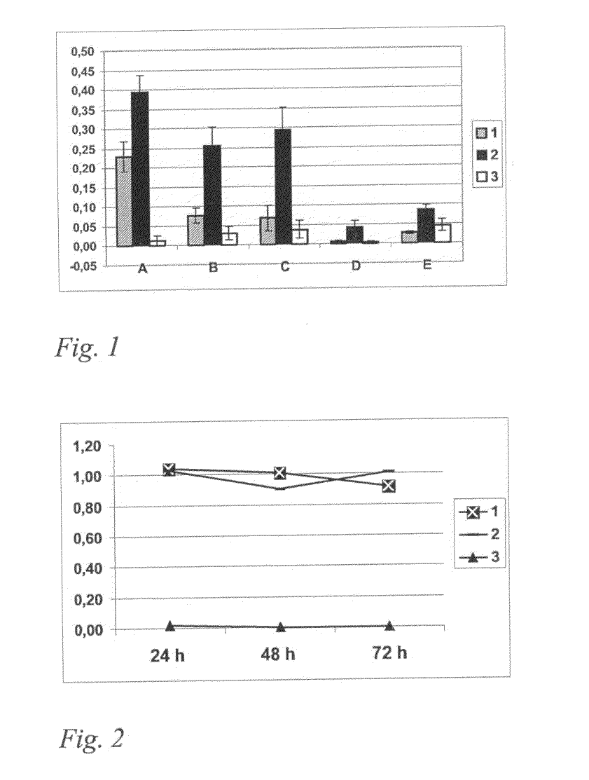

IS257 targeting unit was found non-toxic for all tested cell lines. CuSAO₂ 7.5 μg/ml, used as a positive control, showed 100% cell killing after 1 h treatment. An example of the results is shown as viable count vs. time in FIG. 2, wherein the result is shown as viable count vs. time.

The targeting unit IS257 was added to the NSCLC cell line NCI-H23 in two final concentrations, 50 μg/ml (1) and 500 μg/ml (2), respectively. CuSAO₂ 7.5 μg/ml (3) was used as a positive control for 100% cell killing after 1 h treatment. Monitoring was done at two or three time points (24 h, 48 h, 72 h). Cell killing/viability was analysed using the MTT assay.

### Example 24

### In vivo Cytotoxicity

1 mg of targeting unit IS257 (described in Example 3) was injected i.v. into the tail vein of Athymic nude mice in a volume of 100 μl of sterile physiological saline. The behaviour of mice was observed during 30 min right after injection and during 15 min on the following day (comparison to non-injected mouse). Three mice were taken into this study (plus non-injected controls). Thus, injection of targeting unit IS257 did not have any toxic effect on mice.

### Example 25

### Testing of Immunogenicity of a Targeting Unit of the Invention

Mice and immunization. Female 6- to 8-week old balb/c female mice (Harlan Laboratories, The Netherlands) were used in this study. The targeting unit IS257 (described in Example 3) was dissolved in sterile saline at 0.5 mg/ml and 0.25 mg/ml concentrations. A group of five mice were initially immunized intraperitoneally with 50 μg of targeting unit on day 0. The following immunizations were done with 25 μg of targeting unit on days 14, 28, 56 and 84. Mice were bled from the tail vein on day 0 (preimmune bleed) and thereafter on days 42, 70, and 98 (end point bleed). Blood was collected in tubes and the serum was clarified by centrifugation at 3500 RPM for 7 minutes. Serum samples from mice were pooled and used in a serological assay.

Serological antibody assay. Anti-targeting unit antibody levels in sera from mice immunized with targeting unit IS257 and from non-immunized control mice were assayed by enzyme-linked immunosorbent assays (ELISA) using the targeting agent HP201 (described in Example 6) as capture antigen. Briefly, 150 μl of a 30 μg/ml solution of HP201 in PBS (pH 7.0) was used to coat the wells of Reacti-Bind Maleimide activated clear strip plate (Pierce) overnight at 4° C. The wells were blocked by blocking solution (3% BSA, 0.05% Tween in PBS, pH 7.0) for 1.5 h at 37° C. 1:40 dilution of the test sera from the end point bleed or a control serum in blocking solution were added to the wells in a volume of 150 μl and incubated for 2 h at 37° C. The wells were washed five times with washing buffer (PBS containing 0.05% Tween20) before incubating for 1 h at 37° C. with 150 μl of a 1:1000 dilution of horseradish peroxidase conjugated Affinipure of goat anti-mouse IgG+IgM (Jackson ImmunoResearch Europe Ltd) and detected with a DAB (3,3′-diaminobentzidine) substrate kit for peroxidase (Vector Laboratories).

As a positive control the horseradish peroxidase conjugated Affinipure goat anti-mouse IgG+IgM (10 μg/ml) was used to coat the wells of Reacti-Bind Maleimide activated clear strip plate overnight at 4° C. Development of the signal of the positive controls were done directly with the DAB substrate kit. The plates were read at 405 nm using an ELISA plate reader (ThermoLabsysrems Multiskan EX). The targeting unit IS257 was found to be non-immunogenic in the serological antibody assay, as no anti-targeting unit antibodies could be detected.

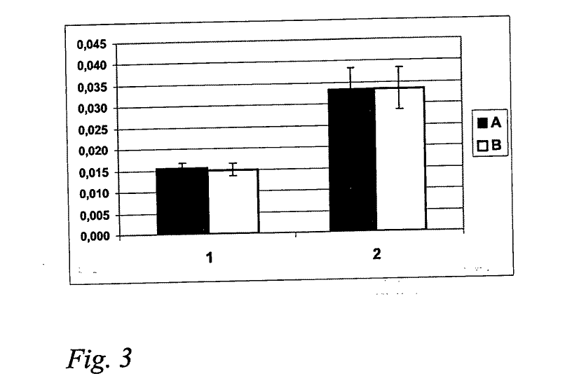

The results are presented in FIG. 3. Mice immunized with a targeting unit of this invention do not develop any immune response. Antibody levels in sera from mice immunized with the targeting unit IS257 (A), and from non-immunized mice (B) were assayed by enzyme-linked immunosorbent assays (ELISA), using HP201 as capture antigen (1). As a positive control, goat anti-mouse antibody was used as a capture antigen (2). The results are shown as measured absorbance at 405 nm.

### LIST OF REFERENCES

Little C D et al. Amplification and expression of the c-myc oncogene in human lung cancer cell lines. Nature 306: 194-196, 1983. PubMed: 6646201
Giard D J et al. In vitro cultivation of human tumors: establishment of cell lines derived from a series of solid tumors. J. Natl. Cancer Inst. 51: 1417-1423, 1973. PubMed: 4357758
Banks-Schlegel S P et al. Intermediate filament and cross-linked envelope expression in human lung tumor cell lines. Cancer Res. 45: 1187-1197, 1985. PubMed: 2578876
Banks-Schlegel S P et al. Intermediate filament and cross-linked envelope expression in human lung tumor cell lines. Cancer Res. 45: 1187-1197, 1985. PubMed: 2578876
Scherer W F et al., Studies on the propagation in vitro of poliomyelitis viruses. IV. Viral multiplication in a stable strain of human malignant epithelial cells (strain HeLa) derived from an epidermoid carcinoma of the cervix.
J Exp Med. 1953 May; 97(5):695-710. No abstract available.

### PMID: 13052828 [PubMed—OLDMEDLINE for Pre1966]

Koga M et al., Cytotoxic, cell agglutinating, and syncytium forming effect of purified lectins from Ricinus communis on cultured cells.

### Gann. 1979 October; 70(5):585-91.

PMID: 520750 [PubMed—indexed for MEDLINE]
Garlanda C et al., Progressive growth in immunodeficient mice and host cell recruitment by mouse endothelial cells transformed by polyoma middle-sized T antigen: implications for the pathogenesis of opportunistic vascular tumors.

### Proc Natl Acad Sci U S A. 1994 Jul. 19; 91(15):7291-5.

PMID: 8041783 [PubMed—indexed for MEDLINE]
O'Connell K A et al., A mouse lymphoid endothelial cell line immortalized by simian virus 40 binds lymphocytes and retains functional characteristics of normal endothelial cells.

### J Immunol. 1990 Jan. 15; 144(2):521-5.

PMID: 2153170 [PubMed—indexed for MEDLINE]
Bregman M G, et al., Difluoromethylornithine enhances inhibition of melanoma cell growth in soft agar by dexamethasone, clone A interferon and retinoic acid.

### Int J Cancer. 1986 Jan. 15; 37(1):101-7.

PMID: 3079741 [PubMed—indexed for MEDLINE]
Smith G P, Scott J K. Libraries of peptide and proteins displayed on filamentous phage. Methods Enzymol. 1993, 217; 228-257.
Elo H O, Lumme P O, Antitumor activity of trans-bis(salicylaldoximato)-copper(II): a novel antiproliferative metal complex.

### Cancer Treat Rep. 1985 September; 69(9):1021-2.

PMID: 4028035 [PubMed—indexed for MEDLINE]

### Boumrah D et al.,

Spacer Molecules in Peptide Sequences: Incorporation into Analogues of Atrial Natriuretic Factor, Tetrahedron, 1997, vol. 53, no. 20, pp. 6977-6992

1. A targeting unit comprising a peptide sequence:
X—R—Y—P—Zₙ
or a pharmaceutically or physiologically or diagnostically acceptable salt thereof, wherein,
X is alanine, serine or homoserine, or a structural or functional analogue thereof;
R is arginine or homoarginine, or a structural or functional analogue thereof; and
Y is arginine, homoarginine, alanine, leucine, serine, homoserine, valine or proline, or a structural or functional analogue thereof;
or
R and Y together constitute a unit that is or comprises at least one optical isomer of arginine or homoarginine, or a structural or functional analogue thereof comprising at least one guanyl or amidino group or related group that has or may obtain a delocalised positive charge through protonation;
P is proline or a structural or functional analogue thereof;
Z is any amino acid residue, and wherein each Zn may be different, similar or identical; and
n is an integer from 0 to 7,
characterized in that that said unit specifically targets tumors.

2. The targeting unit according to claim 1, wherein said tumor is a lung cancer tumor.

3. The targeting unit according to claim 1, wherein said lung cancer is a non-small cell lung cancer tumor.

4. The targeting unit according to claim 1, wherein X is alanine or a structural or functional analogue thereof.

5. The targeting unit according to claim 1, wherein X is serine or a structural or functional analogue thereof.

6. The targeting unit according to claim 1, wherein X is serine or a structural or functional analogue thereof. 6. The targeting unit wherein said lunch cancer is a non-small cell lung cancer tumor, wherein Y is arginine, or a structural or functional analogue thereof.

7. The targeting unit according to claim 1, wherein Y is alanine or a structural or functional analogue thereof.

8. The targeting unit according to claim 1, wherein n is 0-6.

9. The targeting unit according to claim 8, wherein n is 0-5.

10. The targeting unit according to claim 9, wherein n is 0-4.

11. The targeting unit according to claim 10, wherein n is 0-3.

12. The targeting unit according to claim 11, wherein n is 0.

13. The targeting unit according to claim 1, wherein the peptide is linear.

14. The targeting unit according to claim 1, wherein the peptide is cyclic or forms part of a cyclic structure.

15. The targeting unit according to claim 1 selected from the group consisting of the peptides identified by SEQ ID NO. 1-SEQ ID NO. 73.

16. The targeting unit according to claim 15 selected from the group consisting of ARRPKLD (SEQ ID NO. 1), SRRPKLD (SEQ ID NO. 65), ARRP (SEQ ID NO. 66), SRAP (SEQ ID NO. 67), ARAP (SEQ ID NO. 68), SRVP (SEQ ID NO. 69), SRLP (SEQ ID NO. 70), ARLP (SEQ ID NO. 71), ARPP (SEQ ID 72), SRRP (SEQ ID NO. 73).

17. A tumor targeting agent comprising at least one targeting unit of claim 1, directly or indirectly coupled to at least one effector unit.

18. The tumor targeting agent according to claim 17, wherein the effector unit is a directly or indirectly detectable agent or a therapeutic agent.

19. The tumor targeting agent according to claim 18, wherein the detectable agent comprises a chelator, a metal complex, an enriched isotope, radioactive material, a paramagnetic substance, an affinity label, or a fluorescent or luminescent label.

20. The tumor targeting agent according to claim 18, wherein the detectable agent comprises a rare earth metal.

21. The tumor targeting agent according to claim 18, wherein the detectable agent comprises a beta- or an alpha-emittor.

22. The tumor targeting agent according to claim 18, wherein the detectable agent comprises gadoliniumor europium.

23. The tumor targeting agent according to claim 18, wherein the therapeutic agent is selected from the group consisting of cytotoxic, cytostatic and radiation emitting substances.

24. The tumor targeting agent according to claim 23, wherein the therapeutic agent comprises paclitaxel, vinorelbine, irinotecane, cisplatin, carboplatin, doxorubicin, daunorubicin, methotrexate, gemsitabine, alpha- or beta-emitters, or boron.

25. The tumor targeting agent according to claim 17, further comprising at least one optional unit.

26. A diagnostic or pharmaceutical composition comprising at least one targeting unit according to claim 1, or at least one targeting agent which is directly or indirectly coupled to at least one effector unit.

27. Use of a targeting unit according to claim 1, or a targeting agent which is directly or indirectly coupled to at least one effector unit in therapy.

28. Use of a targeting unit according to claim 1, or a targeting agent which is directly or indirectly coupled to at least one effector unit in diagnostics.

29. Use of a targeting unit according to claim 1, or a targeting agent which is directly or indirectly coupled to at least one effector unit for the preparation of a medicament for the treatment of cancer or cancer related diseases.

30. The use according to claim 29, wherein said cancer or cancer related disease is a solid tumor or its metastases.

31. The use according to claim 30, wherein said solid tumor is non-small cell lung cancer or its metastases.

32. Use of a targeting unit according to claim 1, or a targeting agent which is directly or indirectly coupled to at least one effector unit for the preparation of a diagnostic composition for the diagnosis of cancer or cancer related diseases.

33. A method for treating cancer or cancer related diseases, comprising providing to a patient in need thereof a therapeutically effective amount of a pharmaceutical composition according to claim 26.

34. The method according to claim 33, wherein said cancer or cancer related disease is a lung tumor or its metastases.

35. The method according to claim 34, wherein said solid tumor is non-small cell lung cancer or its metastases.

36. A method for diagnosis of cancer or cancer related diseases, comprising providing to a patient in need thereof a diagnostically suitable amount of a diagnostic composition according to claim 26.
